(19) United States
(12) Patent Application Publication
(10) Pub. No.: US 2007/0010015 A1
(43) Pub. Date: Jan. 11, 2007

(54) Complete empty viral particles of infectious bursal disease virus (ibdv), production method thereof and applications of same

(76) Inventors: Rodriguez Aguirre Jose Francisco, Madrid (ES); Maria Dolores Gonzalez de Llano, Madrid (ES); Ana Maria Ona Blanco, Madrid (ES); Fernardo Abaitua Elustondo, Oxted (GB); Antonio Maraver Molina, Madrid (ES); Roberto Clemente Cervera, La Jolla, CA (US); Jose Ruiz Caston, Madrid (ES); Juan Ramon Rodriguez Fernandez-Alba, Madrid (ES)

Correspondence Address: KLARQUIST SPARKMAN, LLP, 121 SW SALMON STREET, SUITE 1600, PORTLAND, OR, 97204, US

(21) Appl. No.: 10/551,877

(22) PCT Filed: Mar. 31, 2004
(86) PCT No.: PCT/ES04/00147

§ 371 Date: Sep. 14, 2006

(30) Foreign Application Priority Data
Mar. 31, 2003 (ES) P200300751

(51) Int. Cl. C12N 15/861; C12N 5/06; C12N 7/00

(52) U.S. Cl. 435/456; 435/348; 435/235.1; 977/802

(57) ABSTRACT

Whole empty viral particles of infectious bursal disease virus (IBDV), which contain all of the antigenically-relevant proteinaceous constituents present in determinant IBDV virions. The whole empty virus particles are readily produced in suitable expression systems to provide capsids that can be used in the production of vaccines against avian disease, e.g., infectious bursitis caused by IBDV, and in the development of gene therapy vectors.

### FIELD OF THE INVENTION

The invention is related to whole empty viral particles of the infectious bursal disease virus (IBDV), with immunogenic activity against IBDV, their production by means of genetic engineering and applications thereof, particularly in the production of animal health vaccines, for example, in the manufacture of vaccines against the avian disease called infectious bursal disease caused by IBDV and in the manufacture of gene therapy vectors.

### BACKGROUND OF THE INVENTION

During the last four decades of the 20^{th} century, the appearance and global spreading of an avian disease called infectious bursal disease (IBD) has occurred. IBD is characterized by the destruction of pre-B lymphocyte populations residing in the bursa of Fabricius of infected animals (Sharma J M et al. 2000. Infectious bursal disease virus of chickens: pathogenesis and immunosuppression. Dev Comp Immunol. 24:223-35). This disease is caused by the infectious bursal disease virus (IBDV) belonging to the Birnaviridae family (Leong J C et al. 2000. Virus Taxonomy Seventh Report of International Committee on Taxonomy of Viruses. Academic Press, San Diego, Calif.). In spite of the implementation of intensive vaccination programs, based on the use of combinations of live and inactivated vaccines, outbreaks of IBD are still reported in all chicken meat-producing countries (van den Berg T P et al. 2000. Infectious bursal disease (Gumboro disease). Rev Sci Tech. 19:509-43).

The virions of the infectious bursal virus lack a lipid envelope, have an icosahedral structure (symmetry T=13) and have a diameter of 65-70 nm (Bottcher B. et al. 1997. Three-dimensional structure of infectious bursal disease virus determined by electron cryomicroscopy. J. Virol. 71:325-30; Castón J. R., et al. 2001. C terminus of infectious bursal disease virus major capsid protein VP2 is involved in definition of the t number for capsid assembly. J. Virol. 75:10815-28). The capsid is formed by a single protein layer containing four different polypeptides called VPX, VP2, VP3 and VP1, respectively. The VPX, VP2 and VP3 proteins are produced by means of proteolytic processing of a precursor, referred to as viral polyprotein, encoded by genomic segment A. The VP1 protein is produced by means of expression of the corresponding gene encoded by segment B.

The viral polyprotein, synthesized as a precursor of 109 kDa, is processed cotranslationally, giving rise to the formation of three polypeptides referred to as VPX, VP3 and VP4. VP4 is responsible for this processing (Birghan C. et al. 2000. A non-canonical Ion proteinase lacking the ATPase domain employs the Ser-Lys catalytic dyad to exercise broad control over the life cycle of a double-stranded RNA virus. Embo J. 19:114-23). VP3 is a polypeptide of 29 kDa forming trimeric subunits coating the inner layer of the capsid. VPX (also known as pVP2) undergoes a second proteolytic processing giving way to the mature form of the protein called VP2. The outer surface of the virions is formed by trimeric subunits constituted of a variable ratio of VPX and VP2 (Chevalier C et al. 2002. The maturation process of pVP2 requires assembly of infectious bursal disease virus capsids. J. Virol. 76:2384-92; Lombardo, E., et al. 1999. VP1, the putative RNA-dependent RNA polymerase of infectious bursal disease virus, forms complexes with the capsid protein VP3, leading to efficient encapsidation into virus-like particles. J. Virol. 73:6973-83). It has been suggested that the conversion of VPX to VP2 is associated with the formation of mature capsids (Chevalier, C., et al. 2002. The maturation process of pVP2 requires assembly of infectious bursal disease virus capsids. J. Virol. 76:2384-92; Martínez-Torrecuadrada, J. L. 2000. Different architectures in the assembly of infectious bursal disease virus capsid proteins expressed in insect cells. Virology. 278:322-31). The polyprotein proteolytic processing sites have been characterized (Da Costa, B., et al. 2002. The capsid of infectious bursal disease virus contains several small peptides arising from the maturation process of pVP2. J. Virol. 76:2393-402; Sánchez, A. B. and Rodríguez, J. F. 1999. Proteolytic processing in infectious bursal disease virus: identification of the polyprotein cleavage sites by site-directed mutagenesis. Virology. 262:190-9), which allows for a reliable expression of the polypeptides of the capsid. The viral RNA-dependent RNA polymerase (RdRp) viral, called VP1, interacts with the VP3 protein, giving rise to a complex facilitating its encapsidation (Lombardo E et al. 1999. VP1, the putative RNA-dependent RNA polymerase of infectious bursal disease virus, forms complexes with the capsid protein VP3, leading to efficient encapsidation into virus-like particles. J. Virol. 73:6973-83; Tacken, M., et al. 2000. Interactions in vivo between the proteins of infectious bursal disease virus: capsid protein VP3 interacts with the RNA-dependent RNA polymerase, VP1. J. Gen. Virol. 81 Pt 1:209-18). The domain of the protein VP3 responsible for this interaction is located in its 16 C-terminal residues (Maraver, A., et al. Identification and molecular characterization of the RNA polymerase-binding motif of the inner capsid protein VP3 of infectious bursal disease virus. J. Virol. 77:2459-2468). The protein VP3 interacts with RNA non-specifically. This reaction does not require the existence of specific sequences in the RNA molecule (Kochan, G., et al. 2003. Characterization of the RNA binding activity of VP3, a major structural protein of IBDV. Archives of Virology 148:723-744). As with that observed with other internal capsid proteins of other viruses, it seems likely that VP3 stabilizes the genomic RNA in the viral particle.

Conventional vaccines used for controlling infectious bursal disease are based on the use of strains, with different degrees of virulence, of the IBDV itself grown in cell culture or in embryonated eggs. The extracts containing the infectious material are subjected to chemical inactivation processes to produce inactivated vaccines, or else are used directly to produce live attenuated vaccines (Sharma, J. M., et al. 2000. Infectious bursal disease virus of chickens: pathogenesis and immunosuppression. Developmental and Comparative Immunology 24:223-235; van den Berg, T. P., et al. 2000. Rev. Sci. Tech. 2000, 19:509-543). This latter type of vaccine has the typical drawbacks associated with the use of live attenuated vaccines, specifically, the risk of mutations reverting the virulence of the virus or causing it to lose its immunogenicity.

Recombinant subunit vaccines containing the IBDV protein VP2 expressed in several expression systems, for example, bacteria, yeasts or baculovirus, usually in fusion protein form, have been disclosed. The results obtained in chicken immunization tests with said vaccines have not been completely satisfactory.

Empty viral capsids or virus-like particles (VLPs,) constitute an alternative to the use of live attenuated vaccines and of recombinant subunit vaccines. VLPs are obtained by self-assembly of the subunits constituting the viral capsid and mimicking the structure and antigenic properties of the native virion, even thought they lack genetic material, as a result of which they are incapable of replicating themselves. Apart from their application for vaccination purposes, VLPs can be used as vectors of molecules of biological interest, for example, nucleic acids, peptides or proteins. By way of illustration, parvovirus VLPs (U.S. Pat. No. 6,458,362) or human immunodeficiency virus (HIV) VLPs (U.S. Pat. No. 6,602,705), can be mentioned.

Morphogenesis is a vital process for the viral cycle requiring successive steps associated to modifications in the polypeptide precursors. As a result, viruses have developed strategies allowing the sequential and correct interaction between each one of its components. One of these strategies, frequently used by icosahedral viruses, is the use of polypeptides coming from a single polyprotein as the base of its structural components. In these cases, the suitable proteolytic processing of such polyprotein plays a crucial role in the assembly process.

The production of several IBDV VLPs by means of expression of the viral polyprotein using different expression systems have been disclosed. In 1997, Vakharia disclosed for the first time, obtainment of IBDV VLPs in insect cells (Vakharia, V. N. 1997. Development of recombinant vaccines against infectious bursal disease. Biotechnology Annual Review 3:151-68). Later, in 1998, the research group to which the inventors belonged proved the possibility of obtaining IBDV VLPs in mammalian cells (Fernández-Arias A et al. 1998. Expression of ORF A1 of infectious bursal disease virus results in the formation of virus-like particles. J. Gen. Virol. 79:1047-54). In 1999, an article was published disclosing the obtaining of IBDV VLPs in insect cells by another research group (Kibenge, F. S., et al. 1999. Formation of virus-like particles when the polyprotein gene (segment A) of infectious bursal disease virus is expressed in insect cells. Can. J. Vet. Res. 63:49-55). A subsequent study, published by the laboratory to which the inventors belong, in collaboration with INGENASA S. A., proved that the morphogenesis of IBDV VLPs in insect cells infected with recombinant baculoviruses expressing the IBDV polyprotein is very ineffective and leads to the major accumulation of abnormal tubular structures (Martínez-Torrecuadrada, J. L., et al. 2000. Different architectures in the assembly of infectious bursal disease virus capsid proteins expressed in insect cells. Virology 278:322-331). These results were subsequently corroborated (Chevalier, C., et al. 2002. The maturation process of pVP2 requires assembly of infectious bursal disease virus capsids. J. Virol. 76:2384-92). In that same article, that group of researchers proved the possibility of obtaining an efficient morphogenesis by means of the expression of a chimeric polyprotein formed by the fusion of the open reading frame (ORF) corresponding to the green fluorescent protein (GFP) and to 3′ end of the open reading phase of the IBDV polyprotein. The expression of this chimeric polyprotein leads to the formation of recombinant IBDV VLPs, containing in their interior a VP3-GFP recombinant fusion protein, different from the one present in the IBDV virions. On the other hand, the results disclosed in this latter research project do not provide information concerning the mechanism responsible for the ineffectiveness of the morphogenetic process of the IBDV VLPs in insect cells.

It is important to stress that all the VLPs disclosed previously lack the VP1 protein, which is present in the IBDV virions. The only reference to the obtaining of IBDV VLPs including VP1 have been carried out by researchers of the laboratory to which the inventors belong (Lombardo, E., et al. 1999. VP1, the putative RNA-dependent RNA polymerase of infectious bursal disease virus, forms complexes with the capsid protein VP3, leading to efficient encapsidation into virus-like particles. J. Virol. 73:6973-83), using the vaccine virus as the vector, which prevents the possible use of said VLPs for vaccination purposes.

The different processes of producing IBDV VLPs previously described suffer from different defects that reduce or prevent their applicability for the generation of vaccines against IBDV, given that:
i) the production of IBDV VLPs in mammalian cells is based on the use of recombinants of the vaccine virus; however, that production system has a very high cost and, as it uses a recombinant virus capable of infecting both mammals and birds, it does not meet the biosafety conditions necessary for its use as a vaccine;
ii) the production of IBDV VLPs in insect cells using conventional expression systems, i.e. recombinant baculoviruses only expressing the viral polyprotein, is very inefficient, leading to practically no production of VLPs;
iii) the production of IBDV VLPs in insect cells by means of the expression of a chimeric polyprotein (formed by the fusion of the ORF corresponding to the GFP at the 3′ end of the ORF corresponding to the IBDV polyprotein) results in the production of IBDV VLPs containing a fusion protein VP3-GFP, which introduces a protein element not present in IBDV virions, of unknown effect and of doubtful applicability in the chicken food chain for human consumption, and
iv) none of the systems described above for the production of IBDV VLPs based on the use of recombinant baculoviruses allows for obtaining IBDV VLPs containing all the antigens present in the IBDV virions.

### SUMMARY OF THE INVENTION

The invention generally is aimed at the problem of providing new effective and safe vaccines against the infectious bursal disease virus (IBDV).

The solution provided by this invention is based on it being possible to obtain IBDV VLPs correctly assembled by means of the simultaneous expression of the viral polyprotein and the IBDV VP1 protein from two independent open reading frames (ORFs) in suitable host cells. In a particular embodiment, the expression of said ORFs is controlled by different promoters. Said IBDV VLPs are formed by auto-assembly of the IBDV VPX, VP2, VP3 and VP1 proteins, whereby they contain all the antigenically relevant protein elements present in the purified and infective IBDV virions and, for this reason, are called “whole IBDV VLPs” in this description. Given that such whole (complete) IBDV VLPs contain all the antigenically relevant protein elements present in the purified and infective virions of IBDV so as to induce an immunogenic or antigenic response, such whole IBDV VLPs can be used for therapeutic purposes, for example, in the development of vaccines, such as vaccines for protecting birds from the infection caused by IBDV or in the development of gene therapy vectors; for diagnostic purposes, etc.

The obtained results clearly show that: (i) IBDV VP3 protein, expressed in insect cells from the expression of the viral polyprotein, undergo a proteolytic processing eliminating the last 13 amino acid residues from its C-terminal end; (ii) the resulting VP3 protein (called VP3T) is incapable of forming oligomers, which produces a virtually complete blocking of the morphogenetic process inducing virtually no production of VLPs; and (iii) the association of the VP3 protein with the VP1 protein protects the first one (VP3) against the proteolytic processing.

These results have allowed for designing a new strategy or process for the efficient production of whole IBDV VLPs and which, unlike the previously described methods, have an effective morphogenesis while at the same time the presence therein of heterologous protein elements inexistent in purified viral particles is prevented. This strategy is based on the use of a gene expression vector or system allowing the coexpression of the viral polyprotein and of the VP1 protein as independent ORFs, which assures the presence of the viral polyprotein and of the IBDV VP1 protein during the assembly process of the whole IBDV VLPs. Under these conditions, the VP3 and VP1 proteins form stable complexes hindering the proteolytic degradation of VP3, assuring its proper functioning, and leading to the incorporation of VP1 in the IBDV VLPs.

In a particular embodiment, said gene expression system is based on the use of a dual recombinant baculovirus simultaneously expressing the viral polyprotein and the IBDV VP1 protein from two independent ORFs controlled by different promoters. In another particular embodiment, such whole IBDV VLPs are obtained as a result of the coinfection of host cells, such as insect cells, with two recombinant baculoviruses, one of them capable of expressing the viral polyprotein and the other one, the IBDV VP1 protein.

The vaccines obtained by using said whole IBDV VLPs have a number of advantages since it prevents the handling of highly infectious material, it prevents the potential risk of the occurrence of new IBDV mutants, and eliminates the use of a live virus on poultry farms, thus preventing the risk of spreading IBDV vaccine strains to the environment.

Consequently, one aspect of the present invention is related to a whole IBDV VLP made up by assembly of the IBDV PVX, VP2, VP3 and VP1 proteins. Said whole IBDV VLP has antigenic or immunogenic activity against the infection caused by IBDV.

A further aspect of this invention is related to a process for the production of said whole IBDV VLPs provided by this invention, based on the gene coexpression of the viral polyprotein and of the IBDV VP1 as two independent ORFs in suitable host cells. In a particular embodiment, the expression of said ORFs is controlled by different promoters.

The gene constructs, expression systems and host cells developed for the implementation of said production process of said whole IBDV VLPs, as well as their use for the production of said whole IBDV VLPs, constitute further aspects of the present invention.

Such whole IBDV VLPs have the ability to immunize animals, particularly, birds, against the avian disease caused by IBDV, as well as the ability to vectorize or incorporate into vehicles molecules of biological interest, for example, polypeptides, proteins, nucleic acids, etc. In a particular embodiment, said whole IBDV VLPs can be used in the development of vaccines to protect birds against the virus causing the avian disease known as infectious bursal disease (IBDV). Virtually any bird, preferably those avian species of economic interest, for example, chickens, turkeys, ganders, geese, pheasants, partridges, ostriches, etc., can be immunized against the infection caused by IBDV with the vaccines provided by this invention. In another particular embodiment, said whole IBDV VLPs can internally incorporate into vehicles products with biological activity, for example, nucleic acids, peptides, proteins, drugs, etc., whereby they can be used in the manufacture of gene therapy vectors.

Therefore, in a further aspect, the present invention is related to the use of said whole IBDV VLPs in the manufacture of medicaments, such as vaccines and gene therapy vectors. Said vaccines and vectors constitute further aspects of the present invention. In a particular embodiment, said vaccine is a vaccine useful for protecting birds from the infection caused by IBDV. In a specific embodiment, said birds are selected from the group formed by chickens, turkeys, ganders, geese, pheasants, partridges, ostriches, preferably chickens.

In another aspect, the invention is related to a process for the production of recombinant baculoviruses useful for the production of whole IBDV VLPs. In a particular embodiment, the recombinant obtained baculoviruses are dual, i.e., the same recombinant baculovirus is able to express in suitable host cells the viral polyprotein and the IBDV VP1 protein from two ORFs, independent and controlled by promoters of different baculoviruses. In another particular embodiment, recombinant baculoviruses are obtained which are able to express in suitable host cells the viral polyprotein from a nucleic acid sequence comprising the ORFs corresponding to the IBDV polyprotein under the control of a promoter, and recombinant baculoviruses able to express in suitable host cells the IBDV VP1 protein from a nucleic acid sequence comprising the ORF corresponding to the IBDV VP1 under the control of a promoter, the same as or different from the one controlling the expression of the viral polyprotein in said recombinant baculoviruses able to express the viral polyprotein. The resulting recombinant baculoviruses (rBVs) constitute a further aspect of the present invention. Such rBVs can be used for the production of whole IBDV VLPs.

### BRIEF DESCRIPTION OF THE FIGURES

FIG. 1 shows the effect of the C-terminal deletion of the IBDV VP3 in the morphogenesis of VLPs. FIG. 1A shows a diagram which graphically represents the genes derived from IBDV expressed by the different recombinants of the vaccine virus [VT7/Poly (Poly), disclosed by Fernández-Arias et al. (Fernández-Arias, A., et al. 1998. Expression of ORF A1 of infectious bursal disease virus results in the formation of virus-like particles. J. Gen. Virol. 79:1047-1054), VT7/PolyΔ907-1012 (PolyΔ907-1012) and VT7VP3 (VP3)] used for checking the effect of the C-terminal end deletion of VP3 in the formation of IBDV VLPs in mammal cells. VT7/Poly (Poly) expresses the whole polyprotein. VT7/PolyΔ907-1012 (PolyΔ907-1012) expresses a deleted form of the polyprotein lacking the 150 C-terminal residues. VT7/VP3 (VP3) expresses the whole VP3 polyprotein. FIG. 1B illustrates the effect of the deletion of the C-terminal end of the IBDV polyprotein on the subcellular distribution of the VPX (pVP2) and VP2 proteins, and includes digital confocal microscopy images obtained from infected cells with the recombinants VT7/Poly (Poly), VT7/PolyΔ907-1012 (PolyΔ907-1012) and VT7/VP3 (VP3), respectively. The cells were fixed at 24 hours post-infection (h.p.i.) and incubated with anti- IBDV VPX/2 (anti-pVP2VP2) rabbit serum and with anti-IBDV VP3 rat serum, followed by incubation with anti-rabbit IgG goat immunoglobulin coupled to Alexa 488 (green) and with anti-rat IgG goat immunoglobulin coupled to Alexa 594 (red). FIG. 1C shows the effect of the deletion of the C-terminal end of the IBDV polyprotein on the assembly of the capsids; cell extracts infected with VT7/Poly (Poly), VT7/PolyΔ907-1012 (PolyΔ907-1012) or coinfected with VT7/PolyΔ907-1012 (PolyΔ907-1012) and VT7/VP3 (VP3) were subjected to fractioning on sucrose gradient. An aliquot of each one of the fractions was placed on an electron microscopy grid, negatively stained and viewed by means of electron microscopy. The images represent the assemblies detected in equivalent fractions of the different gradients.

FIG. 2 shows the results of a comparative analysis by means of Western blot of the IBDV VP3 protein expressed in different expression systems; cell extracts infected with IBDV, VT7/Poly and FB/Poly, respectively, were subjected to sodium dodecylsulfate polyacrylamide gel electrophoresis (SDS-PAGE) and Western blot analysis using anti-IBDV VP3 rabbit serum, followed by incubation with goat immunoglobulin coupled to peroxidase (HRPO: horse radish peroxidase). The signal was detected by means of ECL (Enhanced Chemioluminescence). The position of the immunoreactive bands and those of the molecular weight markers are indicated.

FIG. 3 shows the characterization of C-terminal proteolysis of the IBDV VP3 protein expressed in insect cells. FIG. 1A shows a diagram graphically representing the his-VP3 gene containing a histidine tag fused to the N-terminal end of VP3 expressed by the recombinant baculovirus FB/his-VP3 [occasionally referred to in this description as FB/his-VP3 wt (wild type)]. The sequence corresponding to the histidine tag and the first amino acid residue corresponding to VP3 (underlined) is indicated. Samples corresponding to whole H5 cell extracts (GIBCO), also identified in this description as H5 cells, infected with FB/his-VP3, or to the his-VP3 protein purified by affinity were subjected to SDS-PAGE and Western blot analysis using anti-VP3 rabbit serum (FIG. 1B) or anti-histidine tag (anti-his tag) (FIG. 1C) followed by incubation with goat immunoglobulin coupled to peroxidase. The signal was detected by means of ECL. The position of the immunoreactive bands and those of the molecular weight markers are indicated.

FIG. 4 shows the location of the proteolytic cutting site of the IBDV VP3 protein in insect cells. FIG. 1A is a diagram graphically representing the group of deleted his-VP3 proteins used in the determination of the position of the proteolytic cutting site of the IBDV VP3 protein in insect cells. FIG. 1B shows the result of a Western blot analysis of the different deleted his-VP3 proteins expressed in H5 cells and purified by immobilized metal affinity chromatography (IMAC). H5 cell culture extracts infected with each one of the recombinant baculoviruses were subjected to purification in HiTrap affinity columns (Amersham Pharmacia Biotech). The purified proteins were subjected to SDS-PAGE and Western blot analysis using anti-VP3 rabbit serum, followed by incubation with goat immunoglobulin coupled to peroxidase. The signal was detected by means of ECL. The position of the immunoreactive bands and those of the molecular weight markers are indicated. The arrows indicate the position of the whole protein (F) and the one corresponding to the proteolyzed form (T).

FIG. 5 illustrates that the proteolytic processing of IBDV VP3 in insect cells causes the elimination of a peptide of 1.560 Da from the C-terminal end of his-VP3. H5 cell extracts infected with FB/his-VP3 were subjected to purification by means of IMAC and the resulting purified protein was analyzed by means of mass spectrophotometry in triplicate. FIG. 5A shows the results of one of these experiments. The presence of two polypeptides of 32.004 and 30.444 Da, respectively, was determined, which proves that the proteolytic processing produces the elimination of a peptide of 1.560 Da from the C-terminal end of his-VP3, size which fits with the molecular mass (1.576 Da) corresponding to the 13 C-terminal residues of IBDV VP3, the sequence of which is shown in FIG. 5B.

FIG. 6 shows the effect of the coexpression of IBDV VP1 on the proteolysis of his-VP3. FIG. 6A shows the detection of VP3/VP1 complexes. H5 cells were infected with FB/his-VP3 or with FBD/his-VP3-VP1. At 72 h.p.i., the cells were harvested and the corresponding extracts subjected to purification in HiTrap affinity columns (Amersham Pharmacia Biotech). Samples corresponding to total extracts (T) or to purified proteins were subjected to SDS-PAGE. The gels were subsequently stained with silver nitrate. The position of the molecular weight markers is indicated. FIG. 6B shows the results of a Western blot analysis of extracts of H5 cells infected with FB/his-VP3, FBD/his-VP3-VP1, or coinfected with FB/his-VP3 and FB/VP1, respectively. The infected cells were harvested at 72 h.p.i. and homogenized. The corresponding extracts were subjected to SDS-PAGE and Western blot analysis using anti-VP3 rabbit serum, followed by incubation with goat immunoglobulin coupled to peroxidase. The signal was detected by means of ECL. The position of the molecular weight markers is indicated.

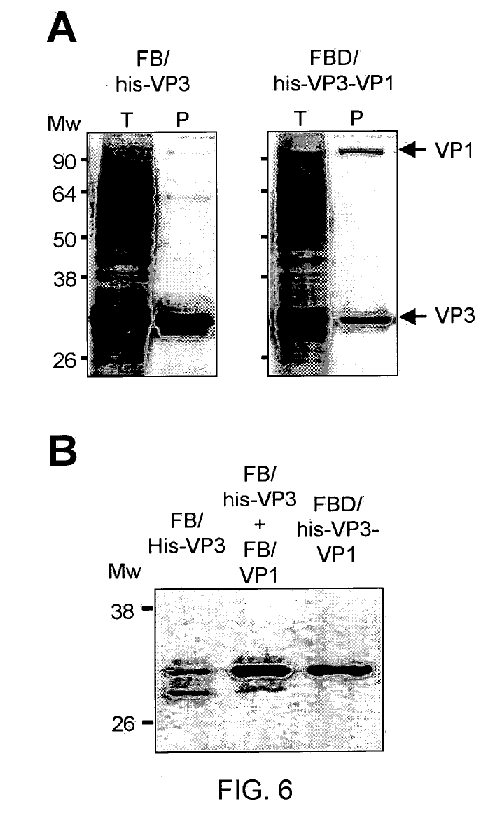

FIG. 7 shows the location of the oligomerization domain. FIG. 7A is a diagram graphically representing the group of deleted his-VP3 proteins used in the determination of the VP3 oligomerization domain position. The deleted regions are indicated with the dotted line. The name of each mutant indicates the location of eliminated amino acid remains in the sequence of the IBDV VP3 protein. FIG. 7B shows the detection of VP3 oligomers. The different his-VP3 deletion proteins, purified by HiTrap affinity columns (Amersham Pharmacia Biotech), were subjected to SDS-PAGE and Western blot analysis using anti-VP3 rabbit serum, followed by incubation with goat immunoglobulin coupled to peroxidase. FIG. 1C shows the results of a Western blot analysis. The samples described in the previous paragraph (FIG. 7B) were subjected to non-denaturing electrophoresis followed by Western blot analysis using anti-VP3 rabbit serum, followed by incubation with goat immunoglobulin coupled to peroxidase. FIG. 7D shows the detection of VP3 oligomers produced by VP3 C-terminal deletion mutants. The purified proteins were subjected to SDS-PAGE and Western blot analysis using anti-VP3 rabbit serum, followed by incubation with goat immunoglobulin coupled to peroxidase. The signal was detected by means of ECL. The position of the molecular weight markers is indicated.

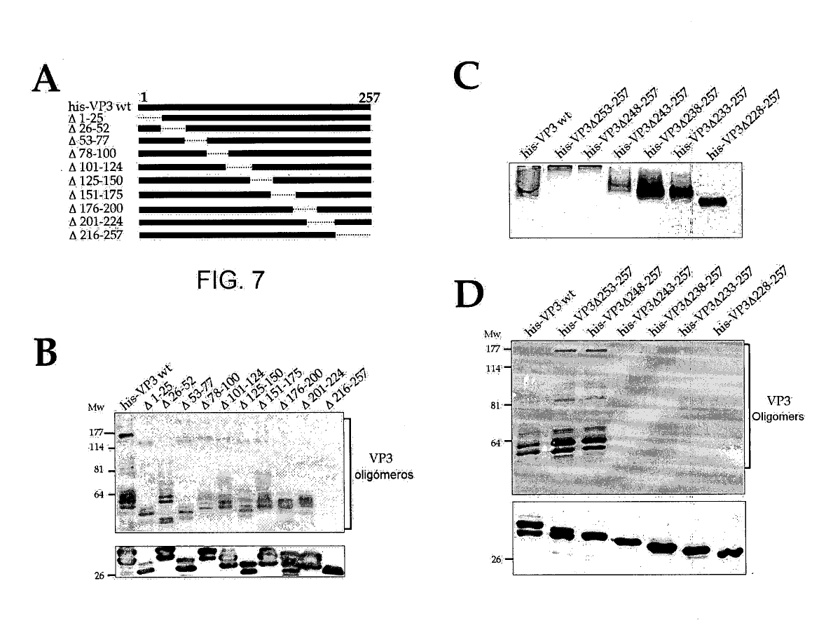

FIG. 8 shows the determination of the effect of the coexpression of IBDV VP1 on the proteolytic processing of IBDV VP3 and the subcellular distribution of the proteins of the capsid. FIG. 8A illustrates the detection of the IBDV VP1 and VP3 proteins accumulated in H5 cells infected with FB/Poly and FBD/Poly-VP1, respectively. Infected cells were harvested at 24, 48 and 72 h.p.i. The samples were subjected to SDS-PAGE and Western blot analysis using anti-VP3 or anti-VP1 rabbit serum, followed by incubation with goat immunoglobulin coupled to peroxidase. The position of the molecular weight markers is indicated. The subcellular distribution of the VPX/2 (pVP2/VP2) and VP3 proteins in cells infected with FB/Poly and FBD/Poly-VP1 was analyzed by confocal microscopy (FIG. 8B). The cells were fixed at 60 h.p.i., and then incubated with anti-VPX rabbit serum (anti-pVP2) and anti-VP3 rat serum followed by incubation with anti-rabbit IgG goat immunoglobulin coupled to Alexa 488 (green) and with anti-rat IgG goat immunoglobulin coupled to Alexa 594 (red). The arrows indicate the position of the viroplasms formed by VPX/2 (pVP2/VP2) and VP3.

FIG. 9 illustrates the characterization of the structures formed by expression of the IBDV polyprotein in cells infected with FB/Poly-VP1. FIG. 9A shows a set of micrographs of the structures obtained in the different fractions. H5 cells were infected with FB/Poly (Poly) or with FBD/Poly-VP1 (Poly-VP1). The cells were harvested at 90 h.p.i. and the corresponding extracts were used for the purification of structures by means of sucrose gradients. After centrifugation, 6 aliquots of 2 ml were taken. One part of the aliquot was placed on a grid, negatively stained with uranyl acetate, and analyzed by means of observation in the electron microscope. Fractions #1 correspond to the bottom of the gradients. Fractions #6, which contained soluble protein and de-assembled structures, are not shown. The bar corresponding to 200 nm. FIG. 9B is a micrograph showing purified VLPs from cells infected with FBD/Poly-VP1. The image corresponds to fraction #5 of the gradient obtained from cells infected with FBD/Poly-VP1. The enlarged boxes show 2 VLPs at a larger amplification. FIG. 9C shows the characterization of the polypeptides present in fraction #5 of both gradients. An aliquot of fraction #5 of each gradient was subjected to SDS-PAGE and Western blot analysis using anti-VP1, anti-VPX (anti-pVP2/VP2) or anti-VP3 rabbit serum, followed by incubation with goat immunoglobulin coupled to peroxidase. The position of VPX (pVP2), VP2, whole VP3 (F) and proteolyzed VP3 (T) is shown.

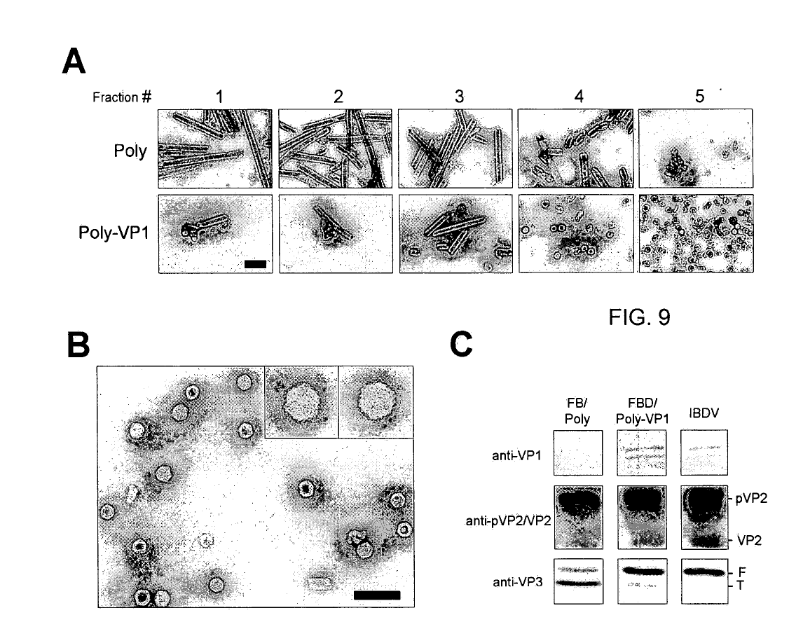

### DETAILED DESCRIPTION OF THE INVENTION

In a first aspect, the invention provides a whole empty viral capsid of the infectious bursal disease virus (IBDV), hereinafter whole IBDV VLP (whole VLPs in plural form) of the invention, characterized in that it contains all the proteins present in purified and infective IBDV virions, specifically the IBDV VPX, VP2, VP3 and VP1 proteins.

The term “IBDV”, as it is used in the present invention, refers to the different IBDV strains belonging to any of the serotypes (1 or 2) known [by way of illustration, see the review carried out by van den Berg, T. P., Eterradossi, N., Toquin, D., Meulemans, G., in Rev Sci Tech 2000 19:509-43].

The terms “viral polyprotein” or “IBDV polyprotein” are generally used in this description and refer to the product resulting from the expression of the A segment of the IBDV genome the proteolytic processing of which gives rise to the VPX (pVP2), VP3 and VP4 proteins, and include the different forms of the polyproteins representative of any of the mentioned IBDV strains [NCBI protein databank], according to the definition carried out by Sánchez and Rodríguez (1999) (Sánchez, A. B. and Rodríguez, J. F. Proteolytic processing in infectious bursal disease virus: identification of the polyprotein cleavage sites by site-directed mutagenesis. Virology. 1999 Sep. 15; 262(1):190-199), as well as proteins substantially homologous to said IBDV polyprotein, i.e., proteins the amino acid sequences of which have a degree of identity regarding said IBDV polyprotein of at least 60%, preferably of at least 80%, more preferably of at least 90% and even more preferably of at least 95%.

The term “IBDV VP1 protein” refers to the product resulting from the expression of segment B of the IBDV genome and includes the different forms of the VP1 proteins representative of any of the mentioned IBDV strains [NCBI protein databank], according to the definition carried out by Lombardo, E., et al. 1999. VP1, The putative RNA-dependent RNA polymerase of infectious bursal disease virus, forms complexes with the capsid protein VP3, leading to efficient encapsidation into virus-like particles. J. Virol. 73:6973-83) as well as proteins substantially homologous to said IBDV VP1 protein, i.e., proteins the amino acid sequences of which have a degree of identity regarding said IBDV VP1 of at least 60%, preferably of at least 80%, more preferably of at least 90% and even more preferably of at least 95%.

The IBDV VPX (pVP2), VP2 and VP3 proteins present in the whole IBDV VLPs of the invention can be any of the VPX, VP2 and VP3 proteins representative of any IBDV strain obtained by proteolytic processing of the viral polyprotein, for example the IBDV Soroa strain VPX, VP2 and VP3 proteins [NCBI, access number AAD30136].

The IBDV VP1 protein present in the whole IBDV VLPs of the invention can be any VP1 protein representative of any IBDV strain, for example, the whole length, Soroa strain VP1 protein, the amino acid sequence of which is shown in SEQ. ID. NO: 2.

In a particular embodiment, the whole IBDV VLPs of the invention have a diameter of 65-70 nm and a polygonal contour indistinguishable from the IBDV virions.

The whole IBDV VLPs of the invention can be obtained by means of the simultaneous expression of said IBDV viral polyprotein and VP1 protein in suitable host cells. Said suitable host cells are cells containing the encoding nucleotide sequence of the IBDV polyprotein under the control of a suitable promoter and the encoding nucleotide sequence of the IBDV VP1 protein under the control of another suitable promoter, either in a single gene construct or in two different gene constructs. In a particular embodiment, said suitable host cells are cells that are transformed, transfected or infected with a suitable expression system, such as (1) an expression system comprising a gene construct, in which such gene construct comprises the nucleotide sequence encoding for the IBDV polyprotein under the control of a promoter and the encoding nucleotide sequence of the IBDV VP1 protein under the control of another promoter different from the one which is operatively bound to the nucleotide sequence encoding the viral polyprotein, or, alternatively, (2) an expression system comprising a first gene construct comprising the nucleotide sequence encoding for the IBDV polyprotein, and a second gene construct comprising the nucleotide sequence encoding for the IBDV VP1 protein, each one of them under the control of a suitable promoter. In a particular embodiment, said host cell is an insect cell and said promoters are baculovirus promoters.

Therefore, in another aspect, the invention is related to a gene construct comprising the nucleotide sequence encoding for said IBDV polyprotein and the nucleotide sequence encoding for said IBDV VP1 protein, in the form of two independent ORFs, the expression of which is controlled by respective different promoters controlling the gene expression of each one of said IBDV viral polyprotein and VP1 protein. Therefore, the invention provides a gene construct comprising (i) a nucleotide sequence comprising the open reading frames corresponding to the polyprotein of the infectious bursal disease virus (IBDV) operatively bound to a nucleotide sequence comprising a first promoter and (ii) a nucleotide sequence comprising the open reading frame corresponding to the IBDV VP1 protein operatively bound to a nucleotide sequence comprising a second promoter, in which such first promoter is different from such second promoter. The use of such different promoters allows the independent and simultaneous control of the gene expression of such IBDV polyprotein and VP1 protein.

A feature of the gene construct provided by this invention is that it comprises the nucleotide sequences encoding for all the protein elements present in the purified and infective IBDV virions, specifically, the VPX, VP2, VP3 and VP1 proteins.

As it is used in this description, the term “ORFs (or open reading frames) corresponding to the IBDV polyprotein” or “ORF (open reading frame) corresponding to the IBDV VP1 protein” includes, in addition to the nucleotide sequences of said ORFs, other ORFs analogous to the same encoding sequences of the IBDV viral polyprotein and of the IBDV VP1. The term “analogous”, as it is used herein, intends to include any nucleotide sequence which can be isolated or constructed on the base of the encoding nucleotide sequence of the viral polyprotein and the IBDV VP1, for example, by means of the introduction of conservative or non-conservative nucleotide replacements, including the insertion of one or more nucleotides, the addition of one or more nucleotides at any end of the molecule, or the deletion of one or more nucleotides at any end or inside of the sequence. Generally, a nucleotide sequence analogous to another nucleotide sequence is substantially homologous to said nucleotide sequence. In the sense used in this description, the expression “substantially homologous” means that the nucleotide sequences in question have a degree of identity, at the nucleotide level, of at least 60%, advantageously of at least 70%, preferably of at least 80%, more preferably of at least 85%, even more preferably of at least 90%, and yet even more preferably of at least 95%.

The promoters which can be used in the implementation of the present invention generally comprise a nucleic acid sequence to which the RNA polymerase is bound so as to begin the mRNA transcription and to express said ORFs corresponding to the viral polyprotein and to the IBDV VP1 protein in suitable host cells. Although virtually any promoter meeting these conditions can be used to implement the present invention, for example, promoters of a viral, bacterial, yeast, animal, plant origin, etc., in a particular embodiment such promoters are viral promoters, for example, baculovirus promoters.

The expression of each one of said nucleotide sequence encoding for said viral polyprotein and IBDV VP1 protein, in the form of two independent ORFs, is controlled by respective different promoters controlling the gene expression of each one of such proteins. In a particular embodiment, the gene expression of such an viral polyprotein and IBDV VP1 protein is carried out in insect cells infected or coinfected with recombinant baculoviruses (rBVs) containing the encoding nucleotide sequences of said proteins, either in a single rBV (dual rBV) or in two rBVs (in which case one of such rBVs contains the encoding sequence of the IBDV polyprotein and the other one, the encoding sequence of the IBDV VP1 protein) under the control of baculovirus promoters.

Virtually any baculovirus promoter can be used as long as it is able to effectively control the expression of the encoding sequence to which it is operatively bound. By way of illustration, the first baculovirus promoter can be the promoter of the p10 protein of the baculovirus Autographa californica nucleopolyhedrovirus (AcMNV), the promoter of the polyhedrin of the AcMNPV baculovirus, etc. and the second baculovirus promoter can be the promoter of the p10 protein of AcMNPV and the promoter of the AcMNPV polyhedrin. More specifically, in a particular embodiment, the first baculovirus promoter is the promoter of the p10 protein of AcMNPV and the second baculovirus promoter is the promoter of the AcMNPV polyhedrin, whereas in another particular embodiment, the first baculovirus promoter is the promoter of the AcMNPV polyhedrin and the second baculovirus promoter is the promoter of the protein 10 of AcMNPV.

In a particular embodiment, the gene construct provided by this invention comprises:
(i) a nucleotide sequence comprising the open reading frames corresponding to the IBDV polyprotein operatively bound to a nucleotide sequence comprising a first promoter of a baculovirus, and
(ii) a nucleotide sequence comprising the ORF corresponding to the IBDV VP1 protein operatively bound to a nucleotide sequence comprising a second promoter of a baculovirus,
   wherein said first and second baculovirus promoters are different.

The use of different baculovirus promoters allows for the independent and simultaneous control of the gene expression of said IBDV polyprotein VP1 protein in insect cells.

In a specific embodiment, the gene construct provided by this invention comprises the encoding sequence of the IBDV polyprotein under the control of a first baculovirus promoter and the encoding sequence of the IBDV VP1 protein under the control of a second baculovirus promoter, different from the first one, such as the gene construct referred to as “Poly-VP1” in this description, comprising the nucleotide sequence identified as SEQ. ID. NO: 1; the Poly-VP1 gene construct contains the encoding sequence of the IBDV polyprotein under the control of the promoter of the AcMNV polyhedrin and the encoding sequence of the IBDV VP1 protein under the control of the promoter of the AcMNV p10 protein.

In another aspect, the invention provides an expression vector or system selected from:
a) an expression system comprising a gene construct provided by this invention, operatively bound to transcription, and optionally translation, control elements, wherein such gene construct includes (i) a nucleotide sequence comprising the ORFs corresponding to the IBDV polyprotein operatively bound to a nucleotide sequence comprising a first promoter and (ii) a nucleotide sequence comprising the ORF corresponding to the IBDV VP1 protein operatively bound to a nucleotide sequence comprising a second promoter, wherein the first promoter is different from the second promoter; and
b) an expression system including (1) a first gene construct, operatively bound to transcription, and optionally translation, control elements, wherein the first gene construct comprises a nucleotide sequence comprising the ORFs corresponding to the IBDV polyprotein operatively bound to a nucleotide sequence including a first promoter, and (2) a second gene construct, operatively bound to transcription, and optionally translation, control elements, wherein the second gene construct includes a nucleotide sequence including the ORF corresponding to the IBDV VP1 protein operatively bound to a nucleotide sequence comprising a second promoter.

In the second case [b)], the first promoter and the second promoter, as they are in different gene constructs, can be equal to or different from one another.

The features of the ORFs corresponding to the IBDV polyprotein and to the IBDV VP1 protein have previously been defined in relation to the gene construct provided by this invention. The promoters which can be used in the expression system provided by this invention have been previously defined in relation to the gene construct provided by this invention. By way of illustration, such promoters can be promoters of a viral, bacterial, yeast, animal, plant origin, etc.

In a particular embodiment, the expression system provided by this invention comprises a gene construct, operatively bound to transcription, and optionally translation, control elements, wherein the gene construct includes (i) a nucleotide sequence including the ORFs corresponding to the IBDV polyprotein operatively bound to a nucleotide sequence including a first baculovirus promoter, such as, for example, the promoter of the AcMNV p10 protein or the promoter of the AcMNV polyhedrin, and (ii) a nucleotide sequence including the ORF corresponding to the IBDV VP1 protein operatively bound to a nucleotide sequence including a second baculovirus promoter, such as, for example, the promoter of the AcMNV p10 protein or the promoter of the AcMNV polyhedrin, wherein the first baculovirus promoter is different from the second baculovirus promoter.

In another particular embodiment, the expression system provided by this invention includes (1) a first gene construct, operatively bound to transcription, and optionally translation, control elements, wherein the first gene construct includes a nucleotide sequence including the ORFs corresponding to the IBDV polyprotein operatively bound to a nucleotide sequence comprising a first baculovirus promoter, such as, for example, the promoter of the AcMNV p10 protein or the promoter of the AcMNV polyhedrin, and (2) a second gene construct, operatively bound to transcription, and optionally translation, control elements, wherein the second gene construct comprises a nucleotide sequence comprising the ORF corresponding to the IBDV VP1 protein operatively bound to a nucleotide sequence including a second baculovirus promoter, such as, for example, the promoter of the AcMNV p10 protein or the promoter of the AcMNV polyhedrin. In this particular embodiment, the first baculovirus promoter and the second baculovirus promoter, as they are in different gene constructs, can be equal to or different from one another.

The transcription, and optionally translation, control elements present in the expression system provided by this invention include the necessary or suitable sequences for the transcription and its suitable control in time and place, for example, beginning and termination signals, cleavage sites, polyadenylation signals, replication origin, transcriptional activators (enhancers), transcriptional silencers (silencers), etc.

Virtually any suitable expression system or vector can be used in the generation of the expression system provided by this invention depending on the conditions and requirements of each specific case. By way of illustration, said suitable expression systems or vectors can be plasmids, bacmids, yeast artificial chromosomes (YACs), bacteria artificial chromosomes (BACs), bacteriophage P1-based artificial chromosomes (PACs), cosmids, viruses, which can further have, if so desired, an origin of heterologous replications, for example, bacterial, so that it may be amplified in bacteria or yeasts, as well as a marker usable for selecting the transfected cells, etc., preferably plasmids, bacmids or viruses.

These expression systems or vectors can be obtained by conventional methods known by persons skilled in the art [Sambrook, J., Fritsch, E. F., and Maniatis, T. (1989). Molecular cloning: a laboratory manual, 2nd ed. Cold Spring Harbor Laboratory] and form part of the present invention. In a particular embodiment, the expression system or vector is a plasmid, such as the plasmid referred to as pFBD/Poly-VP1 in this description, or a bacmid, such as the recombinant bacmid referred to as Bac/pFBD/Poly-VP1 in this description, which contain the previously defined gene construct Poly-VP1, or a virus, such as the recombinant baculovirus (rBV) referred to as FBD/Poly-VP1 in this description, which contains the gene construct Poly-VP1 and expresses during its replication cycle both proteins (polyprotein and IBDV VP1 protein) simultaneously in insect cells, or the rBVs expressing the IBDV polyprotein and the IBDV VP1 protein, separately and simultaneously, when coinfecting insect cells, whole IBDV VLPs being obtained.

In another aspect, the invention provides a host cell containing the encoding nucleotide sequence of the IBDV polyprotein and the encoding nucleotide sequence of the IBDV VP1 protein, each one of them under the control of a suitable promoter allowing the simultaneous and independent control of said IBDV polyprotein and VP1 protein, either in a single gene construct (in which case the promoters bound to each one of said encoding sequences would be different from one another), or in two different gene constructs. Therefore, said host cell can contain either a gene construct provided by this invention or an expression system provided by this invention.

The host cell provided by this invention can be a host cell transformed, transfected or infected with an expression system provided by this invention.

In a particular embodiment, the host cell provided by this invention is a host cell transformed, transfected or infected with an expression system provided by this invention comprising a gene construct, operatively bound to transcription, and optionally translation, control elements, wherein the gene construct includes (i) a nucleotide sequence including the ORFs corresponding to said IBDV polyprotein operatively bound to a nucleotide sequence comprising a first promoter and (ii) a nucleotide sequence comprising the open reading frame corresponding to the IBDV VP1 protein operatively bound to a nucleotide sequence comprising a second promoter, wherein the first promoter is different from the second promoter.

Alternatively, in another particular embodiment, the host cell is a host cell transformed, transfected or infected with an expression system provided by this invention comprising (1) a first gene construct, operatively bound to transcription, and optionally translation, control elements, wherein the first gene construct comprises a nucleotide sequence comprising the ORFs corresponding to said IBDV polyprotein operatively bound to a nucleotide sequence including a first promoter, and (2) a second gene construct, operatively bound to transcription, and optionally translation, control elements, wherein the second gene construct comprises a nucleotide sequence comprising the ORF corresponding to the IBDV VP1 protein operatively bound to a nucleotide sequence comprising a second promoter; in this particular embodiment, the first promoter and the second promoter, as they are in different gene constructs, can be equal to or different from one another.

Although in any of the previously mentioned embodiments, virtually any promoter could be used, it is preferred in practice that such promoters are useful in bacteria, yeasts, viruses, animal cells, for example, in mammal cells, bird cells, insect cells, etc.; in a particular embodiment, the promoters are baculovirus promoters, such as, for example, the promoter of the AcMNV polyhedrin or the promoter of the AcMNV p10 protein.

Virtually any host cell susceptible to being transformed, transfected or infected by an expression system provided by this invention can be used, for example, bacteria, mammal cells, bird cells, insect cells, etc.

In a particular embodiment, the host cell is a bacteria transformed with an expression system provided by this invention including a gene construct provided by this invention comprising (i) a nucleotide sequence comprising the ORFs corresponding to the IBDV polyprotein and (ii) a nucleotide sequence comprising the ORFs corresponding to the IBDV VP1 protein, each one of them operatively bound to a different promoter, such as the gene construct identified as Poly-VP1. A culture of Escherichia coli bacteria strain DH5, transformed with such gene construct Poly-VP1, and identified as DH5-pFBD/Poly-VP1 has been deposited in the Spanish Type Culture Collection (hereinafter, CECT) with deposit number CECT 5777.

Alternatively, the host cell is an insect cell. Insect cells are suitable when the expression system comprises one or more rBVs. The use of rBVs is advantageous due to biosafety issues related to the host range of the baculoviruses, incapable of replicating in other cell types which are not insect cells.

Therefore, in a particular embodiment, the invention provides a host cell, such as an insect cell, infected with an expression system provided by this invention, such as a rBV, comprising a gene construct provided by this invention including (i) a nucleotide sequence including the ORFs corresponding to the IBDV polyprotein and (ii) a nucleotide sequence including the ORF corresponding to the IBDV VP1 protein, each one of them operatively bound to a different baculovirus promoter, such as the gene construct identified as Poly-VP1.

In another particular embodiment, the invention provides host cell, such as an insect cell, coinfected with an expression system including (1) a first rBV comprising a gene construct comprising the ORFs corresponding to the IBDV polyprotein and (2) a second rBV comprising a gene construct comprising the nucleotide sequence comprising the ORF corresponding to the IBDV VP1 protein, each one of the encoding sequences being operatively bound to a baculovirus promoter, equal to or different from one another.

In another aspect, the invention provides a process for producing whole IBDV VLPs of the invention including culturing a host cell provided by this invention containing a nucleotide sequence comprising the ORFs corresponding to said IBDV polyprotein and a nucleotide sequence comprising the ORF corresponding to said IBDV VP1 protein, either in a single gene construct or in two different gene constructs, and simultaneously expressing said viral polyprotein and IBDV VP1 protein, and if so desired, recovering said whole IBDV VLPs of the invention.

In a particular embodiment, the host cell provided by this invention is a cell transformed, transfected or infected with a suitable expression system provided by this invention, such as an expression system comprising a gene construct provided by this invention, wherein such gene construct comprises (i) a nucleotide sequence including the ORFs corresponding to the IBDV polyprotein operatively bound to a nucleotide sequence including a first promoter and (ii) a nucleotide sequence comprising the ORF corresponding to the IBDV VP1 protein operatively bound to a nucleotide sequence comprising a second promoter, wherein either the first promoter is different from the second promoter; or alternatively, with an expression system provided by this invention including (1) a first gene construct comprising a nucleotide sequence comprising the ORFs corresponding to the IBDV polyprotein and (2) a second gene construct comprising a nucleotide sequence comprising the ORF corresponding to the IBDV VP1 protein, each one of the nucleotide sequences comprising the ORFS corresponding to the viral polyprotein and to the IBDV VP1 protein being under the control of respective nucleotide sequences including respective promoters, equal to or different from one another.

Such process therefore includes the simultaneous gene coexpression of the viral polyprotein and IBDV VP1 protein as two independent ORFs. After the simultaneous expression of the viral polyprotein and VP1 protein in such cells, the polyprotein is proteolytically processed and the resulting proteins are assembled and form the whole IBDV VLPs of the invention, made up of VPX, VP2, VP3 and VP1, which can be isolated or withdrawn from the medium and, if desired, purified. The isolation or purification of such whole IBDV VLPs of the invention can be carried out by means of conventional methods, for example, by means of fractioning on sucrose gradients.

Although the host cell to culture can be any of those previously defined, in a particular embodiment, the host cell is an insect cell.

Therefore, in a specific embodiment, the simultaneous gene coexpression of the viral polyprotein and of the IBDV VP1 protein in a suitable host cell, such as an insect cell, is carried out by means of the use of a dual rBV allowing the simultaneous expression of such proteins from two independent ORFs, each one of them under the control of a different baculovirus promoter able to simultaneously and independently control the expression of such proteins in insect cells. In this case, the production of the whole IBDV VLPs of the invention can be carried out by means of a process including, first, the obtaining of a gene expression system made up of a dual rBV containing a gene construct simultaneously including the ORFS corresponding to the viral polyprotein and IBDV VP1 protein, such as the rBV referred to as FBD/Poly-VP1 in this description, or else, alternatively, the obtaining of a rBV containing a gene construct including the ORF corresponding to the IBDV polyprotein and the obtaining of another rBV containing a gene construct comprising the ORF corresponding to the IBDV VP1 protein, followed by the infection of insect cells with the expression system based on such rVB(s), expression of the recombinant proteins and, if so desired, isolation of- the formed whole IBDV VLPs of the invention, and optionally, subsequent purification of the whole IBDV VLPs of the invention.

More specifically, in a particular embodiment, the process for obtaining whole VLPs of the invention is characterized in that the host cell is an insect cell and includes the steps of:
a) preparing an expression system provided by this invention made up of (1) a first recombinant baculovirus comprising a gene construct comprising a nucleotide sequence including the ORFs corresponding to the IBDV polyprotein operatively bound to a baculovirus promoter, such gene construct being operatively bound to transcription, and optionally translation, control elements, and of (2) a second recombinant baculovirus comprising a gene construct including a nucleotide sequence including the ORF corresponding to the IBDV VP1 protein operatively bound to a promoter of a baculovirus, such gene construct being operatively bound to several transcription, and optionally translation, control elements;
b) infecting insect cells with said expression system prepared in step a);
c) culturing the infected insect cells obtained in step b) under conditions allowing the expression of the recombinant proteins and their assembly so as to form whole IBDV VLPs; and
d) if so desired, isolating and optionally purifying such whole IBDV VLPs.

Likewise, in another particular embodiment, the process for obtaining whole VLPs of the invention is characterized in that the host cell is an insect cell and includes the steps of:
a) preparing an expression system made up of a dual recombinant baculovirus including a gene construct including (i) a nucleotide sequence including the ORFs corresponding to the IBDV polyprotein operatively bound to a nucleotide sequence including a first baculovirus promoter, such gene construct being operatively bound to transcription, and optionally translation, control elements, and (ii) a nucleotide sequence comprising the ORF corresponding to the IBDV VP1 protein operatively bound to a nucleotide sequence including a second baculovirus promoter, such gene construct being operatively bound to transcription, and optionally translation, control elements, wherein the baculovirus promoter is different from the second baculovirus promoter;
b) infecting insect cells with said expression system prepared in step a);
c) culturing the infected insect cells obtained in step b) under conditions allowing the expression of the recombinant proteins and their assembly so as to form whole IBDV VLPs; and
d) if so desired, isolating and optionally purifying the whole IBDV VLPs.

The construct of a dual rBV simultaneously allowing expression of the IBDV polyprotein and of the IBDV VP3 protein can be carried out by a person skilled in the art based on that herein described and on the state of the art on this technology (Cold Spring Harbor, N.Y.; Leusch, M. S., Lee, S. C., Olins, P. O. 1995. A novel host-vector system for direct selection of recombinant baculoviruses (bacmids) in Escherichia coli. Gene 160:191-4; Luckow, V. A., Lee, S. C., Barry, G. F., Olins, P. O. 1993. Efficient generation of infectious recombinant baculoviruses by site-specific transposon-mediated insertion of foreign genes into a baculovirus genome propagated in Escherichia coli. J. Virol. 67:4566-79). A rBV containing the gene construct comprising the ORFs corresponding to the IBDV polyprotein and a rBV containing a gene construct including the ORF corresponding to the IBDV VP1 protein can be similarly obtained.

In relation to this, the invention provides a process for obtaining a dual rBV allowing the simultaneous expression of the IBDV polyprotein and of IBDV VP1 protein from two independent ORFs and each one of them controlled by a different baculovirus promoter, in insect cells, including:
a) constructing a plasmid carrier of a gene construct containing (i) a nucleotide sequence including the open reading frames corresponding to the IBDV polyprotein operatively bound to a nucleotide sequence including a first promoter of a baculovirus, and (ii) a nucleotide sequence including the open reading frame corresponding to the IBDV VP1 protein operatively bound to a nucleotide sequence including a second promoter of a baculovirus, wherein the first baculovirus promoter is different from the second baculovirus promoter and they allow the simultaneous control of the gene expression of the polyprotein and IBDV VP1 protein;
b) obtaining a recombinant bacmid, simultaneously allowing the expression during its replicative cycle of the polyprotein and the IBDV VP1 protein under the transcriptional control of the baculovirus promoters, by means of the transformation of competent bacteria with the plasmid obtained in a); and
c) obtaining a dual recombinant baculovirus, allowing the simultaneous expression of the open reading frames corresponding to the polyprotein and the IBDV VP1 protein under the transcriptional control of the baculovirus promoters, by means of the transformation of insect cells with the recombinant bacmid of b).

As used in this description, the term “competent bacteria” refers to bacteria which can contain the genome of a baculovirus, for example, AcMNV, optionally genetically modified, allowing the recombination with donor plasmids.

In a particular embodiment, the process of obtaining dual rBVs is characterized in that:
the first baculovirus promoter sequence comprises the promoter of the AcMNV p10 protein and the second baculovirus promoter sequence includes the promoter of the AcMNPV polyhedrin, or vice versa;
the plasmid obtained in a) is the one identified as pFBD/Poly-VP1 in this description;
the competent bacteria transformed in b) are Escherichia coli DH10Bac;
the recombinant bacmid obtained in b) is the one identified as Bac/pFBD/Poly-VP1 in this description; and
the dual rBV obtained is the one identified as FBD/Poly-VP1.

The dual rBV thus obtained can be used, if so desired, to obtain whole IBDV VLPs of the invention. To that end, insect cells are infected with the dual rBV. Virtually any insect cell can be used; however, in a particular embodiment, the insect cells are H5 cells or Spodoptera frugiperda Sf9 cells.

Alternatively, as previously mentioned, whole VLPs of the invention can be obtained by means of the combined infection (coinfection) of insect cells with a rBV allowing expression of the IBDV polyprotein in insect cells and with a rBV allowing expression of the IBDV VP1 protein in insect cells. Such rBVs can be obtained as previously described. Virtually any insect cell can be used; however, in a particular embodiment, the insect cells are H5 cells or Spodoptera frugiperda Sf9 cells.

Accordingly, in another aspect, the invention is related to a process for the production of rBVs useful for the production of whole IBDV VLPs. In a particular embodiment, the recombinant baculoviruses obtained are dual, i.e., the same recombinant baculovirus is able to express in suitable host cells the viral polyprotein and the IBDV VP1 protein from two independent ORFs and controlled by different baculovirus promoters. The simultaneous expression in the same host cell of the viral polyprotein and IBDV VP1 protein allows the formation of whole IBDV VLPs. In another particular embodiment, recombinant baculoviruses are obtained which are able to express in suitable host cells the viral polyprotein from a nucleic acid sequence comprising the ORFs corresponding to the IBDV polyprotein under the control of a baculovirus promoter and several recombinant baculoviruses able to express in suitable host cells the IBDV VP1 protein from a nucleic acid sequence comprising the ORF corresponding to the VP1 of IBDV under the control of a promoter that is equal to or different from the one regulating the expression of the viral polyprotein in the recombinant baculoviruses able to express the viral polyprotein. The combined infection (coinfection) of suitable host cells, such as insect cells, with the recombinant baculoviruses able to express the viral polyprotein and with the recombinant baculoviruses able to express the IBDV VP1 protein, allows for the simultaneous expression in the coinfected cells of the viral polyprotein and of the IBDV VP1 protein, which allows for the formation of whole IBDV VLPs. The resulting recombinant baculoviruses constitute a further aspect of the present invention.

In another aspect, the invention is related to the use of the gene expression system provided by this invention for the production of whole IBDV VLPs of the invention, which constitute a further aspect of this invention.

The whole IBDV VLPs of the invention can be used to immunize animals, particularly birds, per se or as vectors or vehicles of molecules with biological activity, for example, polypeptides, proteins, nucleic acids, drugs, etc., whereby they can be used for therapeutic or diagnostic purposes. In a particular embodiment, the molecules with biological activity include antigens or immune response inducers in animals or humans to whom they are supplied, or drugs which can be released in their specific action site, or nucleic acid sequences, all being useful in gene therapy and intended for being introduced inside the suitable cells.

Therefore, in another aspect, the invention is related to the use of the whole IBDV VLPs of the invention in the manufacture of medicaments such as vaccines, gene therapy vectors (delivery systems), etc. In a particular embodiment, the medicament is a vaccine intended for conferring protection to animals, particularly birds, against the infectious bursal disease virus (IBDV). In another particular embodiment, the medicament is a gene therapy vector.

In another aspect, the invention provides a vaccine comprising a therapeutically effective amount of whole IBDV VLPs of the invention, optionally together with one or more pharmaceutically acceptable adjuvants and/or vehicles. Such vaccine is useful for protecting animals, particularly birds, against the infectious bursal disease virus (IBDV). In a particular embodiment, such birds are selected from the group formed by chickens, turkeys, geese, ganders, pheasants, partridges and ostriches. In a preferred embodiment, the vaccine provided by this invention is a vaccine useful for protecting chickens from the infection caused by IBDV.

In the sense used in this description, the expression “therapeutically effective amount” refers to the amount of whole IBDV VLPs of the invention calculated for producing the desired effect and will generally be determined, among others, by the characteristics of the whole IBDV VLPs of the invention and the immunization effect to be achieved.

The pharmaceutically acceptable adjuvants and vehicles which can be used in such vaccines are those adjuvants and vehicles known by the persons skilled in the art and normally used in the manufacture of vaccines.

In a particular embodiment, the vaccine is prepared in form of an aqueous solution or suspension in a pharmaceutically acceptable diluent, such as saline solution, phosphate-buffered saline solution (PBS), or any other pharmaceutically acceptable diluent.

The vaccine provided by this invention can be administered by any suitable administration route that results in a protective immune response against the heterologous sequence or epitope used, to which end the vaccine will be formulated in the dosage form suited to the chosen administration route. In a particular embodiment, the administration of the vaccine provided by this invention is carried out parenterally, for example, intraperitoneally, subcutaneously, etc.

The following Examples illustrate the invention and should not be considered limiting of the scope thereof. Example 1 clearly shows that the deletion of the C-terminal end of the IBDV VP3 protein hinders formation of IBDV VLPs, whereas Example 2 describes the generation of a recombinant baculovirus coexpressing the A1 and B1 open reading frames of the IBDV genome, and Example 3 illustrates obtaining whole IBDV VLPs from H5 cells infected with the recombinant baculovirus FBD/Poly-VP1. The materials and methods described below were used to implement the Examples.

### Materials and Methods

Cells and viruses. The recombinant viruses VT7/VP3, VT7/PolyΔ907-1012, FB/Poly, FB/his-VP3 (wt), FB/his-VP3Δ253-257, FB/his-VP3Δ1-25, FB/his-VP3Δ26-52, FB/his-VP3Δ53-77, FB/his-VP3Δ78-100, FB/his-VP3Δ101-124, FB/his-VP3Δ125-150, FB/his-VP3Δ151-175, FB/his-VP3Δ176-200, FB/his-VP3Δ201-224 and FB/his-VP3Δ216-257 were disclosed previously (Fernández-Arias A et al. 1997. The major antigenic protein of infectious bursal disease virus, VP2, is an apoptotic inducer. J. Virol. 71:8014-8; Kadono-Okuda, K., et al. 1995. Baculovirus-mediated production of the human growth hormone in larvae of the silkworm, Bombyx mori. Biochem. Biophys. Res. Commun. 213:389-96; Kochan, G., et al. Characterization of the RNA binding activity of VP3, a major structural protein of IBDV. 2003. Archives of Virology 148:723-744; Martínez-Torrecuadrada, J. L., et al. 2000. Different architectures in the assembly of infectious bursal disease virus capsid proteins expressed in insect cells. Virology. 278:322-31).

The expression experiments were carried out with BSC-1 cells (American Type Culture Collection, ATCC; Catalogue CCL26), H5 [HighFive™ (GIBCO)] and Sf9 cells (GIBCO). The BSC-1 cells were cultured in Eagle modified Dulbecco medium supplemented with 10% fetal bovine serum. The H5 and Sf9 cells were cultured in TC-100 medium (GIBCO) supplemented with 10% fetal bovine serum. The viruses were amplified and titrated following previously disclosed protocols (Lombardo, E., et al. 2000. VP5, the nonstructural polypeptide of infectious bursal disease virus, accumulates within the host plasma membrane and induces cell lysis. Virology. 277:345-57; Martínez-Torrecuadrada, J. L., et al. 2000. Different architectures in the assembly of infectious bursal disease virus capsid proteins expressed in insect cells. Virology. 278:322-31).

The isolate of IBDV used was IBDV Soroa strain.

Generation of recombinant baculoviruses. The previously disclosed plasmid pFB/his-VP3 was used as a mold in the generation, by means of polymerase chain reaction (PCR), of the DNA fragments used in the construction of the plasmid vectors needed for the construction of the recombinant baculoviruses FB/his-VP3Δ248-257, FB/his-VP3Δ243-257, FB/his-VP3Δ238-257, FB/his-VP3Δ233-257, and FB/his-VP3Δ228-257. The PCR reactions were carried out using a common 5′ primer (SEQ. ID. NO: 4) and 3′ primer specific for each mutant (Table 1).

TABLE 1
Generation of deletion mutants of the
terminal carboxy end of His-VP3

| Mutant | Sequence |
|---|---|
| His-VP3Δ248-257 | SEQ. ID. NO: 5 |
| His-VP3Δ243-257 | SEQ. ID. NO: 6 |
| His-VP3Δ238-257 | SEQ. ID. NO: 7 |
| His-VP3Δ233-257 | SEQ. ID. NO: 8 |
| His-VP3Δ228-257 | SEQ. ID. NO: 9 |

After the PCR reactions, the corresponding DNA fragments were purified and digested with the restriction enzymes ApaI and KpnI and ligated to the plasmid pFB/his-VP3 (Kochan, G., et al. 2003. Characterization of the RNA binding activity of VP3, a major structural protein of IBDV. Archives of Virology 148:723-744) previously digested with the same enzymes. The plasmid series generically referred to as pFB/his-ΔVP3 (pFB/his-VP3Δn-n′ more specifically, wherein n and n′ indicate the deleted region borders) containing deletions in the 5′ end of the encoding region of VP3, were thus generated.

The construction of the plasmid vectors required for the generation of the recombinant baculoviruses FB/PolyΔ1008-1012, FB/PolyΔ1003-1012 and FB/PolyΔ998-1012 was carried out by means of the substitution of the Xba I fragment (343 base pairs) with its homologs, containing the desired deletions, deriving from the plasmids FB/his-VP3Δ233-257, FB/his-VP3Δ248-257, and FB/his-VP3Δ243-257, respectively.

The construction of the plasmid vector pFB/VP1 was carried out by means of cloning a DNA fragment, which contains the open reading frame of the gene of the IBDV VP1 protein, from the plasmid pBSKVP1 (Lombardo E et al. 1999. VP1, the putative RNA-dependent RNA polymerase of infectious bursal disease virus, forms complexes with the capsid protein VP3, leading to efficient encapsidation into virus-like particles. J. Virol. 73:6973-83) by means of digestion of the plasmid with the restriction enzyme ClaI, followed by treatment with the Klenow fragment of DNA polymerase I and subsequent treatment with the enzyme NotI. This fragment was subcloned into the vector pFastBac1 (Invitrogen) previously digested with the restriction enzymes Stul and NotI. The resulting plasmid was called pFB/VP1.

The plasmid vectors pFBD/his-VP3-VP1 and pFBD/Poly-VP1 were constructed by means of the insertion of the open reading frames of the genes of the VP3 and VP1 proteins in the vector pFastBacDual (Invitrogen). pFBD/VP1 was generated by means of the insertion of a fragment containing the open reading frame of VP1 obtained by means of digestion with the enzyme NotI, followed by treatment with the Klenow fragment of DNA polymerase I and subsequent treatment with the enzyme XhoI, in the vector pFastBacDual (Invitrogen) previously digested with the enzymes XhoI and PvuII. Next, the plasmid pFB/his-VP3 (Kochan, G., et al. 2003. Characterization of the RNA binding activity of VP3, a major structural protein of IBDV. Archives of Virology 148:723-744) was digested with the enzymes NotI and RsrII, and the resulting fragment containing the open reading frame of his-VP3 was inserted in the plasmid pFBD/VP1 previously digested with the enzymes NotI and RsrII. The resulting plasmid was called pFBD/his-VP3-VP1. Similarly, the open reading frame corresponding to the IBDV polyprotein was isolated from the plasmid pCIneoPoly (Maraver, A., et al. Identification and molecular characterization of the RNA polymerase-binding motif of the inner capsid protein VP3 of infectious bursal disease virus. J. Virol. 77:2459-2468) by means of digestion with the enzymes EcoRI and NotI. The corresponding DNA fragment was cloned into the plasmid pFBD/VP1 previously digested with the enzymes EcoRI and NotI, giving rise to the vector referred to as pFBD/Poly-VP1.

The recombinant baculoviruses described above were generated using the Bac-to-Bac system, following the protocols described by the manufacturer (Invitrogen).

Purification by means of sucrose gradients and characterization of the structures derived from the expression of the IBDV polyprotein. BSC-1 or H5 cells were infected with the described vaccine viruses or recombinant baculoviruses. The infected cells were harvested, lysed and processed as described above (Lombardo, E., et al. 1999. VP1, the putative RNA-dependent RNA polymerase of infectious bursal disease virus, forms complexes with the capsid protein VP3, leading to efficient encapsidation into virus-like particles. J. Virol. 73:6973-83; Castón, J. R., et al. 2001. C terminus of infectious bursal disease virus major capsid protein VP2 is involved in definition of the number for capsid assembly. J. Virol. 75:10815-28).

Electron microscopy. Aliquots of 5 μl of the different fractions of the analyzed sucrose gradients were placed in electron microscopy grids. The samples thus prepared were negatively stained with a 2% uranyl acetate solution. The micrographs were obtained with a Jeol 1200 EXII microscope operating at 100 kV with magnifications of 20,000 or 40,000×.

Purification of his-VP3 fusion proteins and derivatives by means of immobilized metal affinity chromatography (IMAC). H5 or Sf9 cells infected with the different recombinant viruses described were harvested at 72 h.p.i. Alter washing twice in phosphate buffered saline (PBS), the cells were resuspended in lysis buffer (Tris-HCI 50 mM, pH 8.0; NaCl 500 mM) supplemented with protease inhibitors (Complete Mini, Roche) and kept on ice for 20 minutes. Then the samples were subjected to centrifugation at 13,000×g for 10 minutes at 4° C. The corresponding supernatants were subjected to purification by means of IMAC using a resin bound to cobalt (Talon, Clontech Laboratories, Inc., Palo Alto, Calif.) following the manufacturer instructions.

Electrophoresis and Western blot. The protein samples were resuspended in Laemmli buffer (King J & Laemmli UK. 1973. Bacteriophage T4 tail assembly: structural proteins and their genetic identification. J Mol Biol. 1973 Apr. 5;75(2):315-37) and subjected to heating at 100° C. for 5 minutes. The electrophoreses were carried out in 11% polyacrylamide gels. Then the proteins were transferred to nitrocellulose membranes by means of electroblotting. Prior to the incubation with specific antisera, the membranes were blocked by means of incubation for 1 hour at room temperature, with 5% powdered milk diluted in PBS.

Immunofluorescence (IF) and confocal microscopy (CLSM). BSC-1 or H5 cells were grown on slide covers and infected with the recombinant baculoviruses or vaccine viruses. At the post-infection times indicated, the cells were washed two times with PBS and fixed with methanol at −20° C. for 10 minutes. After the fixing, the slide covers were air dried, blocked in a 20% solution of recently born calf serum in PBS 45 minutes at room temperature and incubated with the indicated anti-sera. The samples were viewed by means of epifluorescence using a Zeiss Axiovert 200 microscope equipped with a Bio-Rad Radiance 2100 confocal system. The images were obtained using the Laser Sharp software package programs (Bio-Rad).

Mass spectrophotometry (MS) analysis. The proteins were passed through C-18 ZipTip tips minicolumns (Millipore, Bedford, Mass., USA) and eluted in matrix solution (3,5-dimethoxy-4-hydroxycinnamic acid saturated in aqueous solution of 33% acetonitrile and 0.1% trifluoroacetic acid). An aliquot of 0.7 μl of the resulting mixture was placed in a steel MALDI probe which was subsequently air dried. The samples were analyzed using a Bruker Reflex™ IV MALDI-TOF mass spectrometer (Bruker-Franzen Analytic GmbH, Bremen, Germany) equipped with a SCOUT™ reflector source in positive ion reflector mode using delayed extraction. The acceleration voltage was 20 kV. The equipment was externally calibrated using mass signals corresponding to BSA and BSA dimers ranging from 20-130 m/z.

### EXAMPLE 1

### Deletion of the C-terminal End of the VP3 Protein Eliminates the Formation of IBDV VLPs

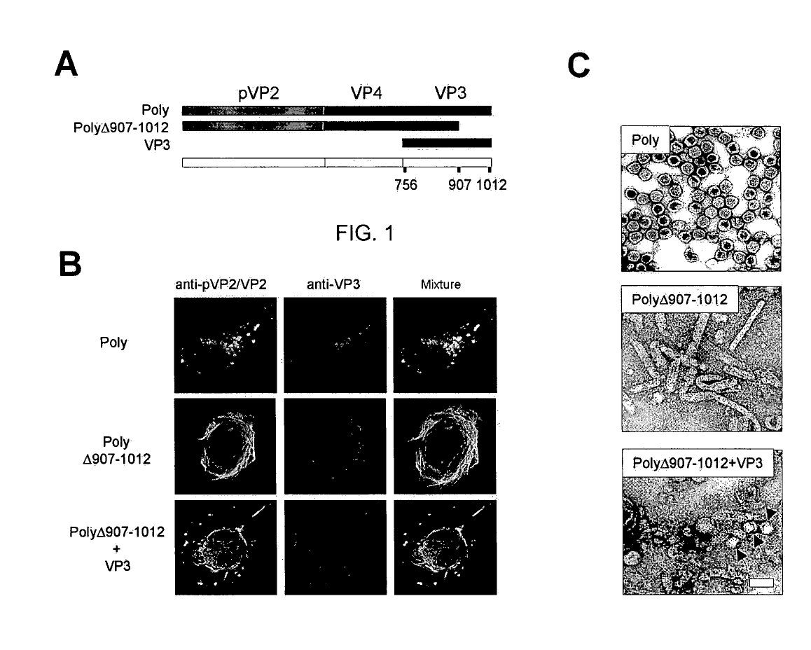

It has recently been disclosed that the C-terminal end of VP3 contains the domain responsible for the interaction of this protein with the VP1 protein (Maraver, A., et al. Identification and molecular characterization of the RNA polymerase-binding motif of the inner capsid protein VP3 of infectious bursal disease virus). As a result, it was decided to analyze the possible role of the C-terminal region of VP3 in the morphogenesis of IBDV VLPs. As a starting ground for this analysis, the recombinant vaccine virus referred to as VT7/PolyΔ907-1012, expressing a deleted form of VP3 lacking the 105 C-terminal end residues (Sánchez Martínez, A. B. 2000. “Caracterización de las modificaciones co y post-traduccionales de la poliproteína del virus de la bursitis infecciosa”. Doctoral Thesis. Universidad Autónoma of Madrid. Facultad of Ciencias Biológicas), was used (FIG. 1A). The SDS-PAGE and Western blot analysis showed that the deletion does not affect the cotranslational proteolytic processing of the polyprotein (Sánchez Martínez, A. B. 2000. Doctoral Thesis cited supra). Expression of the PolyΔ907-1012 protein gives rise to the formation of tubular structures similar to the type I tubules formed in cells infected with IBDV (Kaufer, I., and E. Weiss 1976. Electron-microscope studies on the pathogenesis of infectious bursal disease after intrabursal application of the causal virus. Avian Dis. 20:483-95). The tubular structures formed by expression of PolyΔ907-1012 were detected by means of immunofluorescence using antibodies anti-VPX/2 (anti-pVP2NP2) and anti-VP3 (FIG. 1B), and by means of electron microscopy of fractions obtained by means of purification on sucrose gradients (FIG. 1C). The Western blot analysis confirmed the presence of VPX and VP3 in the tubules.

For the purpose of confirming that the mentioned phenotype was due to the deletion within the region corresponding to VP3, an experiment was carried out coinfecting BSC-1 cells with VT7/PolyΔ907-1012 and VT7VP3. VT7/VP3 is a virus vaccine recombinant expressing the whole VP3 protein (Fernández-Arias, A., et al. 1997. The major antigenic protein of infectious bursal disease virus, VP2, is an apoptotic inducer. J. Virol. 71:8014-8). A confocal microscopy analysis showed that the coexpression of the whole VP3 protein produces a significant reduction in the formation of type I tubules. In the coinfected cells, the subcellular distribution of the VPX/VP3 proteins is characterized by the formation of short tubules and viroplasms similar to those detected in cells infected with the whole polyprotein (FIG. 1B). This observation indicates that the coexpression of the whole VP3 protein partially salvages the ability of the PolyΔ907-1012 protein to form VLPs. The electron microscopy analysis of fractions derived from the coinfection confirmed this hypothesis. Therefore, the top fractions of the gradient were highly enriched in short tubules and quasi-spherical assemblies, called capsoids, with a diameter of 60-70 nm, together with a small proportion of VLPs of polygonal contour (FIG. 1C). The Western blot analysis of the top fractions of the gradient, which contained the highest concentration of capsoids, clearly showed that they contained a larger ratio of whole VP3 protein than of VP3Δ907-1012 protein (data not shown). This result indicated that the incorporation of the whole VP3 protein in these structures is more efficient than that of the deleted form. These results show that the C-terminal end of VP3 plays a fundamental role in the morphogenesis of the IBDV capsid.

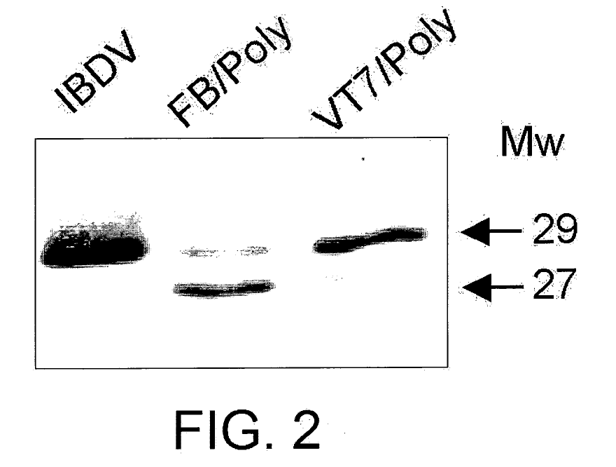

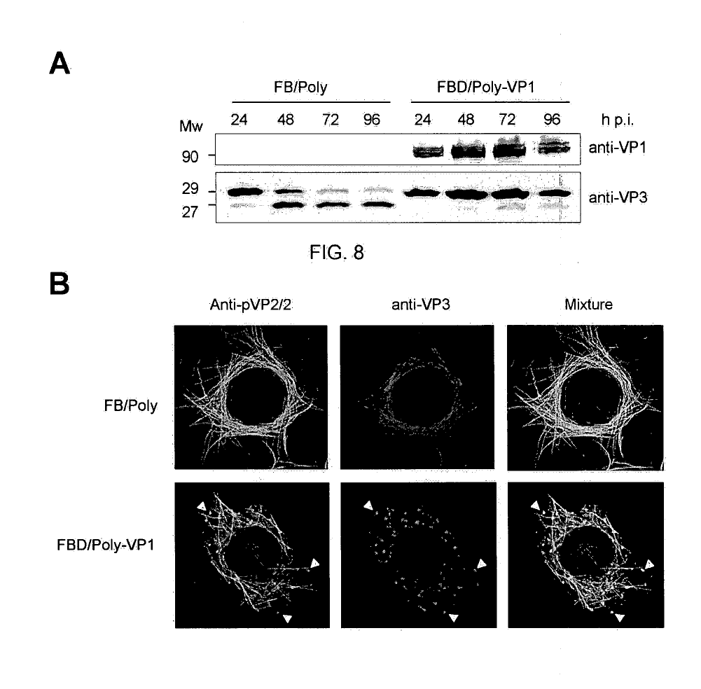

The VP3 protein undergoes a proteolytic processing in insect cells. It has previously been disclosed that the expression of the IBDV polyprotein in insect cells produces the assembly of long tubules formed by VPX trimer hexamers (Da Costa, B., C. Chevalier, C. Henry, J. C. Huet, S. Petit, J. Lepault, H. Boot, and B. Delmas 2002. The capsid of infectious bursal disease virus contains several small peptides arising from the maturation process of pVP2. J. Virol. 76:2393-402; Martínez-Torrecuadrada, J. L., et al. 2000. Different architectures in the assembly of infectious bursal disease virus capsid proteins expressed in insect cells. Virology. 278:322-31). The similarity between the tubules observed in mammalian cells infected with VT7/PolyΔ907-1012 and those detected in insect cells infected with recombinant baculoviruses expressing the whole polyprotein led to the analysis of the condition of the VP3 protein accumulated in insect cells. To that end, cell extracts infected with IBDV, VT7/Poly (Fernández-Arias, A., et al. 1998. Expression of ORF A1 of infectious bursal disease virus results in the formation of virus-like particles. J. Gen. Virol. 79: 1047-54) and FB/Poly, respectively, were analyzed by means of Western blot using anti-VP3 serum (Fernández-Arias. A., et al. 1997. The major antigenic protein of infectious bursal disease virus, VP2, is an apoptotic inducer. J. Virol. 71:8014-8). In cells infected with IBDV and VT7/Poly, the presence of a single band of 29 kDa, the expected size of the whole VP3 protein, was detected by means of Western blot (FIG. 2). On the contrary, in insect cells infected with FB/Poly, the presence of two bands corresponding to polypeptides of 29 and 27 kDa, respectively, was detected by means of Western blot (FIG. 2). An analysis of the time expression showed that even though the appearance of the product of 27 kDa is slightly delayed with regard to the appearance of the product of 29 kDa, it becomes predominant in the later stage of infection (FIG. 8A). A similar analysis carried out in Sf9 cells produced identical results (data not shown). These results show that in insect cells, the VP3 protein undergoes a post-translational modification giving rise to the accumulation of a product of 27 kDa.

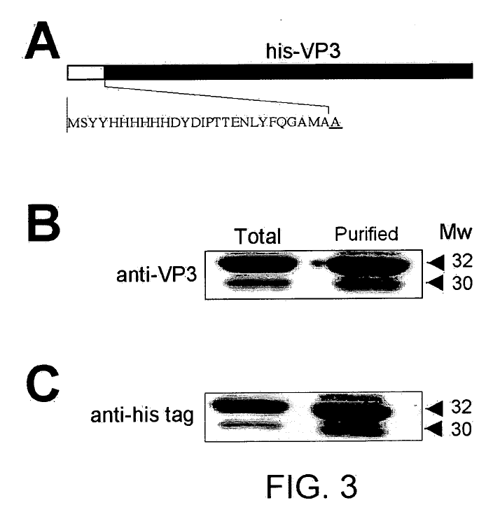

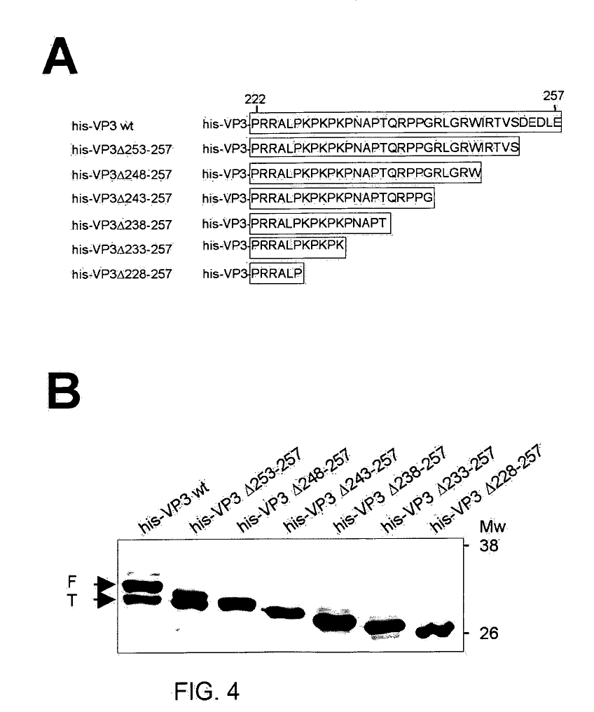

The infection of insect cells with a recombinant baculovirus, FB/his-VP3, expressing a version of VP3 containing a six-histidine residue tag (6xhis), called his-VP3 (FIG. 3A), gives rise to the accumulation of two molecular forms of the protein of 32 and 30 kDa, respectively, similar to those observed in cells infected with FB/Poly (Kochan, G., et al. 2003. Characterization of the RNA binding activity of VP3, a major structural protein of IBDV. Archives of Virology 148:723-744). Therefore, FB/his-VP3 was used as a tool to determine the origin of the smaller VP3 protein. To that end, both total cell extracts infected with FB/his-VP3 and protein purified by means of IMAC were analyzed by means of SDS-PAGE and Western blot using anti-VP3 serum (FIG. 3B) and anti-6xhis (FIG. 3C). As shown in FIG. 3B, the polyprotein of the 30 kDa is present in the purified protein sample, which shows that its N-terminal end remains intact. On the other hand, both the product of 32 kDa and that of 30 kDa are recognized by both antisera (FIGS. 3B and 3C). These results strongly indicate that in insect cells, the VP3 protein undergoes proteolysis, giving rise to a product lacking a fragment of 2 kDa at its C-terminal end. For the purpose of firmly determining this possibility, six recombinant baculoviruses called his-VP3Δ253-257, his-VP3Δ248-257, his-VP3Δ243-257, his-VP3Δ238-257, his-VP3Δ233-257 and his-VP3Δ228-257, respectively (FIG. 4A) were used [they correspond to those defined in the section of Materials and Methods, sub-section Cells and Virus, with an identical nomenclature, but. preceded by “FB/” (indicative of the name of the plasmid used for generating the viruses (pFastBac1)]. These recombinant baculoviruses express a series of deletion forms of VP3 containing a histidine tag. The deletions were generated to progressively eliminate groups of 5 amino acid residues and thus generate a collection with growing deletions at the C-terminal end of the VP3 protein, as shown in FIG. 4A. The expression of these proteins was analyzed by means of Western blot using anti-VP3 serum. As shown in FIG. 4B, the expression of the his-VP3 (his-VP3 wt) whole protein and of the his-VPΔ253-257 mutant protein gave rise to the formation of doublets. On the other hand, the proteins containing deletions of 10 or more residues migrated according to their expected size, giving way to a single band (FIG. 4B). This result shows that the C-terminal end of the VP3 protein is proteolytically processed and that the deletion of the cleavage site prevents proteolysis. The electrophoretic mobility of the his-VP3Δ248-257 protein is slightly less than that of the polypeptides generated by proteolytic processing of his-VP3 and his-VP3Δ253-257, which indicates that the processing occurs in the region located between residues 243 and 248. The C-terminal end of the his-VP3Δ248-257 protein is probably too short so as to allow the recognition on the part of the protease, and therefore it would not undergo proteolytic processing.

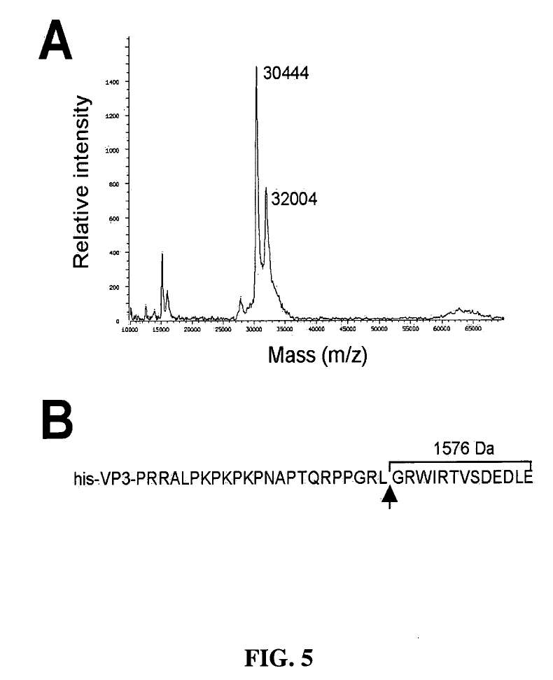

For the purpose of confirming the results obtained with the his-VP3 deletion mutants and precisely establishing the proteolytic cleavage site in the VP3 protein, H5 cell extracts infected with FB/his-VP3 were subjected to purification by means of IMAC. The resulting purified protein was analyzed by means of mass spectrometry. The experiment was repeated three times using independent purifications. The obtained results were similar in all cases (a difference in mass of less than 0.03%). FIG. 5A shows the results of one of these experiments. The presence of two polypeptides of 32,004 and 30,444 Da, respectively, was determined. These results show that the proteolytic processing causes the elimination of a peptide of 1,560 Da from the C-terminal end of his-VP3. This size fits with the molecular mass (1,576 Da) corresponding to the 13 C-terminal residues of VP3 (SEQ. ID. NO: 3) (FIG. 5B).

These results as a whole show that the VP3 protein is proteolytically processed in insect cells between the L244 and G245 residues, giving rise to a polypeptide lacking the 13 C-terminal residues.

### EXAMPLE 2

### Generation of a Recombinant Baculovirus Coexpressing the A1 and B1 Open Reading Frames of the IBDV Genome

### 2.1 Construction of the Plasmid PFBD/VP1

The nucleotide sequence corresponding to the B1 open reading frame of the IBDV genome was obtained from the plasmid pBSKVP1 described above (Lombardo, E., et al. 1999. VP1, the putative RNA-dependent RNA polymerase of infectious bursal disease virus, forms complexes with the capsid protein VP3, leading to efficient encapsidation into virus-like particles. J. Virol. 73:6973-83). The plasmid was purified and subjected to the following enzymatic treatments: i) digestion with the restriction enzyme NotI; ii) incubation with the Klenow fragment of DNA polymerase of E. coli in the presence of dNTPs; and iii) digestion with the restriction enzyme XhoI. Then the corresponding DNA fragment was purified and used for its cloning into the vector pFastBacDual (Invitrogen) previously treated with restriction enzymes XhoI and PvuII. For this, the DNA fragment and the linearized plasmid were incubated in the presence of T4 DNA ligase to generate the plasmid pFBD/VP1.

### 2.2 Construction of the Plasmid pFBD/Poly-VP1

The nucleotide sequence corresponding to the Al open reading frame of the IBDV genome was obtained from the plasmid pCIneoPoly described above (Lombardo, E., et al. 1999. VP1, the putative RNA-dependent RNA polymerase of infectious bursal disease virus, forms complexes with the capsid protein VP3, leading to efficient encapsidation into virus-like particles. J. Virol. 73:6973-83). The plasmid was purified and incubated with the restriction enzymes EcoRI and NotI. The corresponding DNA fragment was purified and incubated with the plasmid pFBD/VP1, previously digested with the restriction enzymes EcoRI and NotI, in the presence of T4 DNA ligase to generate the plasmid pFBD/Poly-VP1. A bacteria culture transformed with said plasmid pFBD/Poly-VP1 has been deposited in the CECT with deposit number CECT 5777.

### 2.3 Obtaining the Bacmid Bac/pFBD/Poly-VP1

This was carried out by means of the transformation of competent bacteria DH10Bac (Invitrogen), positive colony selection in selective medium and purification following the methodology disclosed by Invitrogen (catalog numbers 10359016 and 10608016).

### 2.4 Obtaining the Recombinant Baculovirus FBD/Poly-VP1

The virus was obtained by means of transfection of H5 cells (Invitrogen) with the bacmid Bac/pFBD/Poly-VP1 previously purified following the methodology disclosed by Invitrogen (catalog numbers 10359016 and 10608016).

### EXAMPLE 3

### Obtaining Whole IBDV VLPs from H5 Cells Infected with the Recombinant Baculovirus FBD/Poly-VP1

H5 cell cultures were infected with the recombinant virus FBD/Poly-VP1 (Example 2) using a multiplicity of infection of 5 plaque forming units per cell. The cultures were harvested at 72 hours post-infection (h.p.i). The cells were settled by means of centrifugation (1.500×g for 10 minutes). The cellular sediment was resuspended in PES buffer (PIPES (1,4-piperazine ethanesulfonic acid) 25 mM, pH 6.2, NaCl 150 mM, CaCl₂ 20 mM). Then the cells were homogenized by means of three consecutive freezing/thawing cycles (−70° C./+37° C.). The corresponding homogenate was centrifuged (10,000×g for 15 minutes at 4° C.). The resulting supernatant was harvested and used for the purification of the VLPs. To that end, a centrifuge tube with a 25% sucrose cushion (weight/volume), diluted in PES buffer of 4 ml, was prepared, depositing 8 ml of supernatant thereon. The tube was centrifuged (125,000×g for 3 hours at 4° C.). The resulting sediment was resuspended in 1 ml of PES buffer. Then a continuous 25-50% sucrose gradient in PES buffer was prepared in a centrifuge tube, depositing the resuspended sediment thereon. The tube was centrifuged (125,000×g for 1 hour at 4° C.). Then the gradient was fractioned into aliquots of 1 ml.

The different aliquots were analyzed by means of transmission electron microscopy. To that end, a volume of 5 μl of each sample was placed on a microscope grid. The samples were negatively stained with an aqueous solution of 2% uranyl acetate. A Jeol 1200 EXII microscope operating at 100 kV and at a nominal magnification of 40,000× was used. This analysis showed the presence of whole VLPs structurally identical to the IBDV virions in the analyzed samples.

For the purpose of determining the protein composition of the VLPs detected by means of electron microscopy, the samples were analyzed by means of Western blot. To that end, the samples were subjected to polyacrylamide gel electrophoresis. The gels were subsequently transferred to nitrocellulose and incubated with anti-VPX/2 antibodies (anti-pVP2VP2), anti-VP3 and anti-VP1. The results showed the presence of the VPX, VP2, VP3 and VP1 proteins in the fractions containing VLPs.

Microorganism Deposit

A culture of the bacteria derived from DH5, carrier of a plasmid containing the IBDV polyprotein-VP1 genetic construction (pFBD/Poly-VP1), DH5-pFBD/poly-VP1, has been deposited in the Spanish Culture Type Collection (CECT), University of Valencia, Research Building, Burjasot Campus, 46100 Burjasot, Valencia, Spain, on Mar. 8, 2003, with deposit number CECT 5777.

1. A gene construct comprising (i) a nucleotide sequence comprising the open reading frames corresponding to the polyprotein of the infectious bursal disease virus (IBDV) operatively bound to a nucleotide sequence comprising a first promoter and (ii) a nucleotide sequence comprising the open reading frame corresponding to the IBDV VP1 protein operatively bound to a nucleotide sequence comprising a second promoter, wherein said first promoter is different from said second promoter.

2. A gene construct according to claim 1, wherein said first promoter is a viral promoter and said second promoter is a viral promoter different from said first promoter.

3. A gene construct according to claim 1, comprising:
(i) a nucleotide sequence comprising the open reading frames corresponding to the polyprotein IBDV operatively bound to a nucleotide sequence comprising a first promoter of a baculovirus, and
(ii) a nucleotide sequence comprising the open reading frame corresponding to the IBDV VP1 protein operatively bound to a nucleotide sequence comprising a second promoter of a baculovirus,
wherein said first baculovirus promoter is different from said second baculovirus promoter.

4. A gene construct according to claim 3, wherein said first baculovirus promoter is selected from the group consisting of the promoter of the p10 protein of the baculovirus Autographa californica nucleopolyhedrovirus (AcMNV) and the promoter of the polyhedrin of the baculovirus AcMNPV.

5. A gene construct according to claim 3, wherein said second baculovirus promoter is selected from the group consisting of the promoter of the AcMNPV p10 protein and the promoter of the AcMNPV polyhedrin.

6. A gene construct according to claim 3, wherein said first baculovirus promoter is the promoter of the AcMNPV p10 protein and said second baculovirus promoter is the promoter of the AcMNPV polyhedrin; or wherein said first baculovirus promoter is the promoter of the AcMNPV polyhedrin and said second baculovirus promoter is the promoter of the AcMNPV p10 protein.

7. A gene construct according to claim 1, comprising the nucleotide sequence of SEQ ID NO: 1.

8. An expression system selected from:
a) an expression system comprising a gene construct according to claim 1, operatively bound to transcription, and optionally translation, control elements; and
b) an expression system comprising (1) a first gene construct, operatively bound to transcription, and optionally translation, control elements, wherein said first gene construct comprises a nucleotide sequence comprising the open reading frames corresponding to the IBDV polyprotein operatively bound to a nucleotide sequence comprising a first promoter, and (2) a second gene construct, operatively bound to transcription, and optionally translation, control elements, wherein said second gene construct comprises a nucleotide sequence comprising the open reading frame corresponding to the IBDV VP1 protein operatively bound to a nucleotide sequence comprising a second promoter.

9. An expression system according to claim 8, comprising a gene construct, operatively bound to transcription, and optionally translation, control elements, wherein said gene construct comprises (i) a nucleotide sequence comprising the open reading frames corresponding to the IBDV polyprotein operatively bound to a nucleotide sequence comprising a first baculovirus promoter and (ii) a nucleotide sequence comprising the open reading frame corresponding to the IBDV VP1 protein operatively bound to a nucleotide sequence comprising a second baculovirus promoter, wherein said first baculovirus promoter is different from said second baculovirus promoter.

10. An expression system according to claim 8, comprising (1) a first gene construct, operatively bound to transcription, and optionally translation, control elements, said first gene construct comprising a nucleotide sequence comprising the open reading frames corresponding to the IBDV polyprotein operatively bound to a nucleotide sequence comprising a first baculovirus promoter, and (2) a second gene construct, operatively bound to transcription, and optionally translation, control elements, said second gene construct comprising a nucleotide sequence comprising the open reading frame corresponding to the IBDV VP1 protein operatively bound to a nucleotide sequence comprising a second baculovirus promoter.

11. An expression system according to claim 10, wherein said first baculovirus promoter and said second baculovirus promoter are equal to or different from one another.

12. An expression system according to claim 8, selected from plasmids, bacmids, yeast artificial chromosomes (YACs), bacteria artificial chromosomes (BACs), P1 bacteriophage-based artificial chromosomes (PACs), cosmids and viruses, which can optionally contain a heterologous replication origin.

13. A host cell containing:
(A) a gene construct comprising (i) a nucleotide sequence comprising the open reading frames corresponding to the polyprotein of the infectious bursal disease virus (IBDV) operatively bound to a nucleotide sequence comprising a first promoter and (ii) a nucleotide sequence comprising the open reading frame corresponding to the IBDV VP1 protein operatively bound to a nucleotide sequence comprising a second promoter, wherein said first promoter is different from said second promoter;
(B) the gene construct of (A) operatively bound to transcription, and optionally translation, control elements; or
(C) an expression system comprising (1) a first gene construct, operatively bound to transcription, and optionally translation, control elements, wherein said first gene construct comprises a nucleotide sequence comprising the open reading frames corresponding to the IBDV polyprotein operatively bound to a nucleotide sequence comprising a first promoter, and (2) a second gene construct, operatively bound to transcription, and optionally translation, control elements, wherein said second gene construct comprises a nucleotide sequence comprising the open reading frame corresponding to the IBDV VP1 protein operatively bound to a nucleotide sequence comprising a second promoter.

14. A cell transformed, transfected or infected with an expression system according to claim 8.

15. A cell according to claim 13, selected from the group consisting of animal cells and bacteria.

16. A cell according to claim 15, characterized in that it is the bacteria identified as DH5-pFBD/Poly-VP1 which is deposited in the CECT with deposit number CECT 5777.

17. A cell according to claim 15, selected from the group consisting of insect cells, bird cells and mammal cells.

18. A dual recombinant baculovirus simultaneously expressing the IBDV polyprotein and the IBDV VP1 protein from (i) a nucleotide sequence comprising the open reading frames corresponding to the IBDV polyprotein operatively bound to a nucleotide sequence comprising a first baculovirus promoter, and from (ii) a nucleotide sequence comprising the open reading frame corresponding to the IBDV VP1 protein operatively bound to a nucleotide sequence comprising a second baculovirus promoter, wherein said first baculovirus promoter is different from said second baculovirus promoter.

19. A process for the production of whole empty viral capsids of IBDV, comprising use of:
(A) an expression system comprising a gene construct including (i) a nucleotide sequence comprising the open reading frames corresponding to the polyprotein of the infectious bursal disease virus (IBDV) operatively bound to a nucleotide sequence comprising a first promoter and (ii) a nucleotide sequence comprising the open reading frame corresponding to the IBDV VP1 protein operatively bound to a nucleotide sequence comprising a second promoter, wherein said first promoter is different from said second promoter, wherein said gene construct is operatively bound to transcription, and optionally translation, control elements;
(B) an expression system comprising (1) a first gene construct, operatively bound to transcription, and optionally translation, control elements, wherein said first gene construct comprises a nucleotide sequence comprising the open reading frames corresponding to the IBDV polyprotein operatively bound to a nucleotide sequence comprising a first promoter, and (2) a second gene construct, operatively bound to transcription, and optionally translation, control elements, wherein said second gene construct comprises a nucleotide sequence comprising the open reading frame corresponding to the IBDV VP1 protein operatively bound to a nucleotide sequence comprising a second promoter; or
(C) a dual recombinant baculovirus simultaneously expressing the IBDV polyprotein and the IBDV VP1 protein from (i) a nucleotide sequence comprising the open reading frames corresponding to the IBDV polyprotein operatively bound to a nucleotide sequence comprising a first baculovirus promoter, and from (ii) a nucleotide sequence comprising the open reading frame corresponding to the IBDV VP1 protein operatively bound to a nucleotide sequence comprising a second baculovirus promoter, wherein said first baculovirus promoter is different from said second baculovirus promoter.

20. A process for the production of whole empty viral capsids of the infectious bursal disease virus (IBDV) [whole IBDV VLPs], comprising culturing a host cell as claimed in claim 13, and if desired, recovering said whole IBDV VLPs.

21. A process according to claim 20, wherein said host cell is a cell transformed, transfected or infected with an expression system comprising a gene construct comprising (i) a nucleotide sequence comprising the open reading frames corresponding to said IBDV polyprotein operatively bound to a nucleotide sequence comprising a first promoter and (ii) a nucleotide sequence comprising the open reading frame corresponding to said IBDV VP1 protein operatively bound to a nucleotide sequence comprising a second promoter, wherein said first promoter is different from said first promoter.

22. A process according to claim 20, wherein said host cell is a cell transformed, transfected or infected with an expression system comprising a gene construct comprising (1) a first gene construct comprising a nucleotide sequence comprising the open reading frames corresponding to said IBDV polyprotein and (2) a second gene construct comprising a nucleotide sequence comprising the open reading frame corresponding to said IBDV VP1 protein, each one of said nucleotide sequences comprising the ORFS corresponding to the viral polyprotein and to the IBDV VP1 protein being under the control of respective nucleotide sequences comprising respective promoters, equal to or different from one another.

23. A process according to claim 21, wherein said host cell is an insect cell.

24. A process according to claim 20, wherein said host cell is an insect cell, comprising the steps of:
a) preparing an expression system made up of a dual recombinant baculovirus comprising a gene construct comprising (i) a nucleotide sequence comprising the open reading frames corresponding to the IBDV polyprotein operatively bound to a nucleotide sequence comprising a first baculovirus promoter, said gene construct being operatively bound to transcription, and optionally translation, control elements, and (ii) a nucleotide sequence comprising the open reading frame corresponding to the IBDV VP1 protein operatively bound to a nucleotide sequence comprising a second baculovirus promoter, said gene construct being operatively bound to transcription, and optionally translation, control elements, wherein said baculovirus promoter is different from said second baculovirus promoter;
b) infecting insect cells with said expression system prepared in step a);
c) culturing the infected insect cells obtained in step b) under conditions allowing the expression of the recombinant proteins and their assembly to form whole IBDV VLPs; and
d) if desired, isolating and optionally purifying said whole IBDV VLPs.

25. A process according to claim 20, wherein said host cell is an insect cell, comprising the steps of:
a) preparing an expression system made up of (1) a first recombinant baculovirus comprising a gene construct comprising a nucleotide sequence comprising the open reading frames corresponding to the IBDV polyprotein operatively bound to a baculovirus promoter, said gene construct being operatively bound to transcription, and optionally translation, control elements, and of (2) a second recombinant baculovirus comprising a gene construct comprising a nucleotide sequence comprising the open reading frame corresponding to the IBDV VP1 protein operatively bound to a promoter of a baculovirus, said gene construct being operatively bound to transcription, and optionally translation, control elements;
b) infecting insect cells with said expression system prepared in step a);
c) culturing the infected insect cells obtained in step b) under conditions allowing the expression of the recombinant proteins and their assembly to form whole IBDV VLPs; and
d) if desired, isolating and optionally purifying said whole IBDV VLPs.

26. Whole empty capsids of the infectious bursal disease virus (IBDV) [whole IBDV VLPs], obtained according to the process of claim 20.

27. Whole empty capsids of the infectious bursal disease virus (IBDV) [whole IBDV VLPs], characterized by containing the VPX, VP2, VP3 and VP1 proteins of IBDV.

28. A therapeutic composition comprising whole empty capsids of the infectious bursal disease virus (IBDV) [whole IBDV VLPs] containing the VPX, VP2, VP3 and VP1 proteins of IBDV.

29. A method of combating avian infectious bursal disease, comprising administering to an avian subject a vaccine comprising whole empty capsids of the infectious bursal disease virus (IBDV) [whole IBDV VLPs] containing the VPX, VP2, VP3 and VP1 proteins of IBDV.

30. A gene therapy vector including whole empty capsid of the infectious bursal disease virus (IBDV) [whole IBDV VLP] containing the VPX, VP2, VP3 and VP1 proteins of IBDV.

31. A vaccine comprising a therapeutically effective amount of whole empty capsids of IBDV [whole IBDV VLPs], containing the VPX, VP2, VP3 and VP1 proteins of IBDV, optionally combined with one or more pharmaceutically acceptable adjuvants and/or vehicles.

32. A vaccine according to claim 31, for protecting birds from the infection caused by IBDV.

33. A vaccine according to claim 32, wherein said birds are selected from the group formed by chickens, turkeys, geese, ganders, pheasants, partridges and ostriches.

34. A vaccine for protecting chickens from an infection caused by infectious bursal disease virus (IBDV), comprising a therapeutically effective amount of whole empty capsids of IBDV, whole IBDV VLPs, containing the VPX, VP2, VP3 and VP1 proteins of IBDV, optionally combined with one or more pharmaceutically acceptable adjuvants and/or vehicles.

35. A process for obtaining a dual recombinant baculovirus allowing the simultaneous expression in insect cells of the polyprotein of the infectious bursal disease virus (IBDV) and of the IBDV VP1 protein from two independent open reading frames and each one of them controlled by a different baculovirus promoter, comprising:
a) constructing a plasmid carrying a gene construct containing (i) a nucleotide sequence comprising the open reading frames corresponding to the IBDV polyprotein operatively bound to a nucleotide sequence comprising a first promoter of a baculovirus, and (ii) a nucleotide sequence comprising the open reading frame corresponding to the IBDV VP1 protein operatively bound to a nucleotide sequence comprising a second promoter of a baculovirus, wherein said first baculovirus promoter is different from said second baculovirus promoter;
b) obtaining a recombinant bacmid, allowing the simultaneous expression during its replicative cycle of the polyprotein and the IBDV VP1 protein under transcriptional control of said baculovirus promoters, by means of the transformation of competent bacteria with the plasmid obtained in a); and
c) obtaining a dual recombinant baculovirus, allowing the simultaneous expression of the open reading frames corresponding to the polyprotein and the IBDV VP1 protein under transcriptional control of said baculovirus promoters, by means of transformation of insect cells with the recombinant bacmid of b).

36. A process according to claim 35, wherein:
said first baculovirus promoter is the promoter of the AcMNV p10 protein and said second baculovirus promoter is the promoter of the AcMNPV polyhedrin, or vice versa;
the plasmid obtained in a) is the one identified as pFBD/Poly-VP1;
the competent bacteria of b) are E. coli DH10Bac;
the recombinant bacmid obtained in b) is the one identified as Bac/pFBD/Poly-VP1; and
the recombinant baculovirus obtained is the one identified as FBD/Poly-VP1.

37. A process according to claim 35, further comprising the infection of insect cells with the dual recombinant baculovirus obtained in step c).

38. A process according to claim 37, wherein said insect cells are H5 or Spodoptera frugiperda Sf9 cells.


SEQUENCE LISTING (9 sequences)

SEQ ID NO: 1 - DNA, 10909 bp, Artificial Sequence
  Synthetic Construct
  gene (3)..(3041): Open reading frame of IBDV polyprotein in reverse complementary strand
  promoter (3083)..(3211): AcMNV polyhedrin promoter
  promoter (3230)..(3351): AcMNV p10 promoter
  CDS (3388)..(6027): Open reading frame of IBDV VP1 protein
  polyA_site (6068)..(6331)
  gene (6901)..(7434): Gentamicin resistance gene
  misc_feature (7501)..(7725): Minitransposon Tn7R
  gene (8787)..(9647): Ampicillin resistance gene
  misc_feature (9854)..(10234): F1
  misc_feature (10418)..(10583): Minitransposon Tn7L
gctcactcaaggtcctcatcagagacggtcctgatccagcggcccagccgaccagggggtctctgtgttggagcattgggttttggcttgggctttggtagagcccgcctgggattgcgatgcttcatctccatcgcagtcaagagcagatctttcatctgttcttggtttgggccacgtccatggttgatttcatagactttggcaacttcgtctatgaaagcttggggtggctctgcctgtcctggagccccgtagatcgacgtagctgcccttaggatttgttcttctgatgccaaccggctcttctctgcatgcacgtagtctagatagtcctcgtttgggtccggtatttctcgtttgttctgccagtactttacctggcctgggcttggccctcggtgcccattgagtgctacccattctggtgttgcaaagtagatgcccatggtctccatcttctttgagatccgtgtgtctttttccctctgtgcttcctctggtgtggggccccgagcctccactccgtagcctgctgtcccgtacttggccctttgcgacttgctgcctgcttgtggtgcgtttgcaagaaaatttcgcatccgatgggcgttcgggtcgctgagtgcgaagttggccatgtcagtcacaatcccattctcttccagccacatgaacacactgagtgcagattggaatagtgggtccacgttggctgctgcttccattgctctgacggcactctcgagttcgggggtctctttgaactctgatgcagccatggcaaggtggtactggcgtcctgcattgggtggaaggtatggtaggttgaggtaggggagcctgtcccagtcgcgtggattgtgagggaaacgtttgatgaacgttgcccagttgggcccggtgtttacatcgaatgctccgggaccagccaacctaaggccaagtcggtgtgcagtagcgagcttggtgcttctaaagcttactttctcaatctcgccacaagcattgagggctcccgtcatagccacatggattgggactttgggtcgaaacacatccatgtaagctatggctagatttccactgtttcccacaataggaggtatgggatctttggacagcataatgctgtcgtcccagacatcatctattgggacaacggtgtagtctctcccagtctccagtggaagtaccccatctggagcatatccatagactctgtgtccagagagagttcgtatgaaggatcctctttgagatggaggttggaggtcttctcgcacgccttcaatgacagcaaacattttgctgttcaatgctttgggtgtcatggcgtcttccactgtcgtaataaccacagggaatagcgtggcaccctctcttaacacgcagtcgaggttgtgtgcaccgcggagtaccccaggtgaagcaagaatcccgtcgactacgggattctggggcacctggaatagattcgcgactacctcgtaccccttgtcggcggcgagagtcagctgccttatgcggcctgaggcagctcttgcttttcctgacgcggctcgagcagttcctgaagcggcctgggcctcatcgcccagcaggtagtctacaccttccccaattgcatgggctaggggagcggcaggtgggaacaatgtggagaccaccggcacagctatcctccttatggcccggattatgtctttgaagccgaatgctcctgcaatcttcaggggagagttgaggtcggccacctccatgaagtattcacgaaagtcagtgtactcccttgttggccagacggtcttgatgccaagacggtccctctcactcagtatcaattttgtgtagttcatggctcctgggtcaaatcggccgtattctgtaaccaggttctttgctagttcaggatttgggatcagctcgaagttgctcaccccagcgaccgtaacgacggatcctgttgccactctttcgtaggccactagcgtgacgggacggagggcccctggatagttgccaccatggatcgtcactgctaggctccctcttgccgaccatgacatctgatcccctgcctgaccaccacttttggaggtcactatctccagtttgatggatgtgattggctgggttatctcgtttgttggaatcacaagattgaatggcataaggttgtcggtgccggtcgtcagcccattgtttgcggccacagccctggtgattaccgttgtcccatcaaagcctatgaggtagatggtggcgcccagtacaaggccgtggacgcttgttcgaaacacgagctctcccccaacgctgaggcttgtgatggcatcaatgttggctgagaacagtgtgattgttaccccacctggttggtactgtgatgagaattggtaatcatcggctgcagttatggtgtagactctgggcctgtcactgctgtcacatgtggctaccatttttgggtcaagccctattgcgggaatggggtcaccaagcctcacatacccaagatcatatgatgtgggtaagctgaggacggtgaccccttcccctactaggacgttcccaattttgtcgttgatgttggctgttgcagacatcaacccattgtagctaacatctgtcagttcactcaggcttccttggaaggtcacggcgtttatggtgccgtttagtgcataaacgccaccaggaagtgtgcttgacctcactgtgagactccgactcactagcctgcagtagttgtaactggccggtaggttctgggcagtcaggagcatctgatcgaacttgtagttcccattgccctgcagtgtgtagtgagcacccacaattgagccagggaatccagggaaaaagacaattagccctgaccctgtgtcccccacagtcaaattgtaggtcgaggtctctgacctgagagtgtgcttctccagggtgtcgtccggaatggacgccggtccggttgttggcatcagaaggctccgtatgaacggaacaatctgctgggtttgatctgacaggtttgtcatcgatgcgatcgaattccgcgcgcttcggaccgggatccgcgcccgatggtgggacggtatgaataatccggaatatttataggtttttttattacaaaactgttacgaaaacagtaaaatacttatttatttgcgagatggttatcattttaattatctccatgatctattaatattccggagtatacggacctttaattcaacccaacacaatatattatagttaaataagaattattatcaaatcatttgtatattaattaaaatactatactgtaaattacattttatttacaatcactcgacgaagacttgatcacccgggatctcgaggtcgacggtatcgatgagtgacgttttcaatagtccacaggcgcgaagcacgatctcagcagcgttcggcataaagcctactgctggacaagacgtggaagaactcttgatccctaaagtttgggtgccacctgaggatccgcttgccagccctagtcgactggcaaagttcctcagagagaacggctacaaagttttgcagccgcggtctctgcccgagaatgaggagtatgagaccgaccaaatactcccagacttagcatggatgcgacagatagaaggggctgttttaaaacccactctatctctccctattggagatcaggagtacttcccaaagtactacccaacacatcgccctagcaaggagaagcccaatgcgtacccaccagacatcgcactactcaagcagatgatttacctgtttctccaggttccagaggccaacgagggcctaaaggatgaagtaaccctcttgacccaaaacataagggacaaggcctatggaagtgggacctacatgggacaagcaaatcgacttgtggccatgaaggaggtcgccactggaagaaacccaaacaaggatcctctaaagcttgggtacacttttgagagcatcgcgcagctacttgacatcacactaccggtaggcccacccggtgaggatgacaagccctgggtgccactcacaagagtgccgtcacggatgttggtgctgacgggagacgtagatggcgactttgaggttgaagattaccttcccaaaatcaacctcaagtcatcaagtggactaccatatgtaggtcgcaccaaaggagagacaattggcgagatgatagctatatcaaaccagtttctcagagagctatcaacactgttgaagcaaggtgcagggacaaaggggtcaaacaagaagaagctactcagcatgttaagtgactattggtacttatcatgcgggcttttgtttccaaaggctgaaaggtacgacaaaagtacatggctcaccaagacccggaacatatggtcagctccatccccaacacacctcatgatctccatgatcacctggcccgtgatgtccaacagcccaaataacgtgttgaacattgaagggtgtccatcactctacaaattcaacccgttcagaggagggttgaacaggatcgtcgagtggatattggccccggaagaacccaaggctcttgtatatgcggacaacatatacattgtccactcaaacacgtggtactcaattgacctagagaagggtgaggcaaactgcactcgccaacacatgcaagccgcaatgtactacatactcaccagagggtggtcagacaacggcgacccaatgttcaatcaaacatgggccacctttgccatgaacattgcccctgctctagtggtggactcatcgtgcctgataatgaacctgcaaattaagacctatggtcaaggcagcgggaatgcagccacgttcatcaacaaccacctcttgagcacgctagtgcttgaccagtggaacttgatgagacagcccagaccagacagcgaggagttcaaatcaattgaggacaagctaggtatcaactttaagattgagaggtccattgatgatatcaggggcaagctgagacagcttgtcctccttgcacaaccagggtacctgagtgggggggttgaaccagaacaatccagcccaactgttgagcttgacctactagggtggtcagctacatacagcaaagatctcgggatctatgtgccggtgcttgacaaggaacgcctattttgttctgctgcgtatcccaagggagtagagaacaagagtctcaagtccaaagtcgggatcgagcaggcatacaaggtagtcaggtatgaggcgttaaggttggtaggtggttggaactacccactcctgaacaaagcctgcaagaataacgcaggcgccgctcggcggcatctggaggccaaggggttcccactcgacgagttcctagccgagtggtctgagctgtcagagttcggtgaggccttcgaaggcttcaatatcaagctgaccgtaacatctgagagcctagccgaactgaacaagccagtaccccccaagcccccaaatgtcaacagaccagtcaacactgggggactcaaggcagtcagcaacgccctcaagaccggtcggtacaggaacgaagccggactgagtggtctcgtccttctagccacagcaagaagccgtctgcaagatgcagttaaggccaaggcagaagccgagaaactccacaagtccaagccagacgaccccgatgcagactggttcgaaagatcagaaactctgtcagaccttctggagaaagccgacatcgccagcaaggtcgcccactcagcactcgtggaaacaagcgacgcccttgaagcagttcagtcgacttccgtgtacacccccaagtacccagaagtcaagaacccacagaccgcctccaacccggttgttgggctccacctgcccgccaagagggccaccggtgtccaggccgctcttctcggagcaggaacgagcagaccaatggggatggaggccccaacacggtccaagaacgccgtgaaaatggccaaacggcggcaacgccaaaaggagagccgccaatagccatgaggcggccctgatgcatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttgtcgagactgcaggctctagattcgaaagcggcc

SEQ ID NO: 2 - protein, 879 aa, Artificial Sequence
  Synthetic Construct
MSDVFNSPQARSTISAAFGIKPTAGQDVEELLIPKVWVPPEDPLASPSRLAKFLRENGYKVLQPRSLPENEEYETDQILPDLAWMRQIEGAVLKPTLSLPIGDQEYFPKYYPTHRPSKEKPNAYPPDIALLKQMIYLFLQVPEANEGLKDEVTLLTQNIRDKAYGSGTYMGQANRLVAMKEVATGRNPNKDPLKLGYTFESIAQLLDITLPVGPPGEDDKPWVPLTRVPSRMLVLTGDVDGDFEVEDYLPKINLKSSSGLPYVGRTKGETIGEMIAISNQFLRELSTLLKQGAGTKGSNKKKLLSMLSDYWYLSCGLLFPKAERYDKSTWLTKTRNIWSAPSPTHLMISMITWPVMSNSPNNVLNIEGCPSLYKFNPFRGGLNRIVEWILAPEEPKALVYADNIYIVHSNTWYSIDLEKGEANCTRQHMQAAMYYILTRGWSDNGDPMFNQTWATFAMNIAPALVVDSSCLIMNLQIKTYGQGSGNAATFINNHLLSTLVLDQWNLMRQPRPDSEEFKSIEDKLGINFKIERSIDDIRGKLRQLVLLAQPGYLSGGVEPEQSSPTVELDLLGWSATYSKDLGIYVPVLDKERLFCSAAYPKGVENKSLKSKVGIEQAYKVVRYEALRLVGGWNYPLLNKACKNNAGAARRHLEAKGFPLDEFLAEWSELSEFGEAFEGFNIKLTVTSESLAELNKPVPPKPPNVNRPVNTGGLKAVSNALKTGRYRNEAGLSGLVLLATARSRLQDAVKAKAEAEKLHKSKPDDPDADWFERSETLSDLLEKADIASKVAHSALVETSDALEAVQSTSVYTPKYPEVKNPQTASNPVVGLHLPAKRATGVQAALLGAGTSRPMGMEAPTRSKNAVKMAKRRQRQKESRQ

SEQ ID NO: 3 - protein, 13 aa, Infectious bursal disease virus
GRWIRTVSDEDLE

SEQ ID NO: 4 - DNA, 37 bp, Artificial Sequence
  Synthetic Construct
gggggaattcatggcatcagagttcaaagagaccccc

SEQ ID NO: 5 - DNA, 31 bp, Artificial Sequence
  Synthetic Construct
cgcgggtaccttaccagcggcccagccgacc

SEQ ID NO: 6 - DNA, 33 bp, Artificial Sequence
  Synthetic Construct
cgcgggtaccttaaccagggggtctctgtgttg

SEQ ID NO: 7 - DNA, 33 bp, Artificial Sequence
  Synthetic Construct
cgcgggtaccttatgttggagcattgggttttg

SEQ ID NO: 8 - DNA, 31 bp, Artificial Sequence
  Synthetic Construct
cgcgggtaccttattttggcttgggctttgg

SEQ ID NO: 9 - DNA, 31 bp, Artificial Sequence
  Synthetic Construct
cgcgggtaccttatggtagagcccgcctggg